(19) United States
(12) Patent Application Publication
(10) Pub. No.: US 2023/0349803 A1
(43) Pub. Date: Nov. 2, 2023

(54) CELL CLASSIFICATION ALGORITHMS, AND USE OF SUCH ALGORITHMS TO INFORM AND OPTIMISE MEDICAL TREATMENTS

(71) Applicant: Oxford Nanoimaging Limited, Oxford Oxfordshire (GB)

(72) Inventors: Andras G. Miklosi, Oxford Oxfordshire (GB); James H. Felce, Oxford Oxfordshire (GB); Bo Jing, Oxford Oxfordshire (GB); Tyler Ralston, Oxford Oxfordshire (GB)

(73) Assignee: Oxford Nanoimaging Limited, Oxford Oxfordshire (GB)

(21) Appl. No.: 18/025,614

(22) PCT Filed: Sep. 10, 2021
(86) PCT No.: PCT/EP2021/074954

§ 371 Date: Mar. 9, 2023

(30) Foreign Application Priority Data
Sep. 10, 2020 (GB) 2014223.8

(51) Int. Cl. G01N 15/02; G01N 21/64; G01N 33/50; G01N 33/574

(52) U.S. Cl. CPC G01N 15/02; G01N 21/6458; G01N 33/505; G01N 33/57484; G01N 2015/0288

(57) ABSTRACT

The invention provides a method of investigating the spatial organisation of proteins in or on cells, and the use of that spatial organisation information to inform decisions about medical interventions, especially in relation to cancer treatments including CAR-T therapy. The method involves detecting one or more species of proteins on each of the plurality of cells; obtaining respective spatial coordinates of the detected proteins within the plurality of cells; detecting boundaries of the plurality of cells; and constructing a data vector based on the obtained spatial coordinates and the detected boundaries.

### TECHNICAL FIELD

The present application relates to methods for classifying cells, and ways to employ such classification methods to inform and optimise medical interventions, especially in the context of cell therapies.

### BACKGROUND

Novel classes of drugs (biologics) and recently developed cellular therapies rely on the modulation and modification of the patient's own cells, such as cells of the immune system, to target and interact with diseased cells such as cancer cells.

These cell therapies such as immunotherapies have led to spectacular outcomes in the treatment of a growing number of different diseases, including various malignancies, and there is great potential for their broader therapeutic application, in particular, in cancer therapy. However, using current approaches, the same cell therapy can often show spectacular success in one patient and no benefit at all, or worse, serious side-effects, when administered to a different individual suffering from apparently the same condition. At the heart of this problem is an inadequate understanding of the molecular mechanisms underpinning these therapies, which may lead to the manufacture and/or administration of suboptimal or inappropriate cell therapies, and unreliable and inconsistent patient diagnostic processes being used in the clinic.

Improved technologies are, therefore, required, which provide a better understanding and prediction of the interaction between the target cells of individual patients and effector cells provided by different potential cell therapies.

### SUMMARY

According to a first aspect of the present invention, there is provided a method of investigating a plurality of cells, comprising: detecting one or more species of proteins on each of the plurality of cells; obtaining respective spatial coordinates of the detected proteins within the plurality of cells; detecting boundaries of the plurality of cells; and constructing a data vector based on the obtained spatial coordinates and the detected boundaries.

In some implementations, constructing the data vector further comprises: evaluating a spatial distribution based on the obtained spatial coordinates.

In some implementations, constructing the data vector comprises: performing a spatial distribution analysis algorithm such that the obtained spatial coordinates are partitioned into one or more clusters at a predetermined number of length scales. At each length scale, each cluster comprises the spatial coordinates of the detected proteins within an area corresponding to the length scale.

In such implementations, constructing the data vector may comprise: performing a spatial distribution analysis algorithm such that the obtained spatial coordinates are partitioned into one or more clusters at a predetermined number of length scales, wherein at each length scale, each cluster comprises the spatial coordinates of the detected proteins within an area corresponding to the length scale; and determining a set of properties for the clusters at each of the length scales; wherein the data vector comprises the set of properties determined for the clusters at each of the length scales.

In some implementations, obtaining the boundaries comprises: obtaining an optical image of the plurality of cells; performing a segmentation algorithm on the optical image of the plurality of cells; and extending a border obtained by the segmentation algorithm by a predetermined distance.

In some implementations, the constructed data vector comprises at least one measure of the localisation distribution of detected proteins within the cells. This localisation distribution may be one or more of: (a) the number density of localisations of the spatial coordinates of the detected proteins within the cells; (b) the distance between localisation across multiple types of proteins; or (c) Ripley's K function.

Preferably, the constructed data vector comprises at least one measure of the cluster characteristics of the cells. This may be any of the measures 2.a-2.v set out in the detailed description, and/or the number of clusters. For example, the measure may be an average (mean/median) and optionally variation (e.g. variance, standard deviation etc.) of one or more of (a) the cluster radius/diameter at multiple length scales, (b) 35 cluster area at multiple length scales; (c) cluster density at multiple length scales; (d) cluster shape (e.g. circularity) at multiple length scales; and (e) number of localisations per cluster at multiple length scales. Suitably, said “multiple length” scales comprise at least 2, at least 3, at least 4, or at least 5 different length scales. For example, the length scale may be all or a subset of 10 nm, 50 nm, 100 nm, 500 nm and 1000 nm.

In some implementations, the data vector includes a measure of cell-cell interactions. This would apply, for example, where the plurality of cells being investigated is in the context of a tumor or tissue sample. The measure of cell-cell interactions may be, for example (a) an average (mean/median) and optionally variation (e.g. variance, standard deviation etc.) of the distance between cells; (b) an average (mean/median) and optionally variation (e.g. variance, standard deviation etc.) of the distance between cells of different types; (c) neighbouring cell cluster colocalization; and (d) Ripley's K function distribution of cells.

Optionally, the data vector may comprise or consist of (1) the number of clusters; (2) an average (mean and/or median) and optionally variance/SD of the area of clusters; (3) an average (mean and/or median) and optionally variance/SD of the distance between clusters; and (4) an average (mean and/or median) and optionally variance/SD of the number of localisations per cluster. Preferably, all of (1)-(4) are provided across multiple length scales, e.g. 10 nm, 50 nm, 100 nm, 500 nm and 1000 nm.

In some implementations, constructing the data vector further comprises: performing colocalization analysis on an overlapping area between any two of the plurality of cells.

In some implementations, the method further comprises constructing a feature vector by performing a dimension reduction analysis on the constructed data vector, wherein a first dimension of the feature vector is larger than two and smaller than a second dimension of the data vector.

In some implementations, the dimension reduction analysis comprises Principal Component Analysis (PCA) such that the feature vector comprises a first number of principal components obtained from the data vector, and wherein the first dimension is the first number.

Suitably, the method of investigating the plurality of cells comprises a labelling step, prior to said detecting one or more species of proteins on each of the plurality of cells. The labelling step may involve incubating the cells with a fluorescent marker specific to the protein of interest. Alternatively, the labelling step may involve modifying the cells so as to express the protein of interest labelled with a fluorescent protein. In such implementations, the step of detecting one or more species of proteins on each of the plurality of cells and obtaining respective spatial coordinates consists or comprises of carrying out single molecule localisation microscopy, for example using dSTORM or fPALM.

In some implementations, there is provided method of classifying a plurality of cells of a patient into a plurality of types of reference cells, comprising: investigating the plurality of cells of the patient and the reference cells aforementioned to obtain a first feature vector for the plurality of cells of the patient and a second feature vector of the reference cells; evaluating a probability distance metric between the first feature vector and the second feature vector; and determining whether the patient is classified into one of the types.

In some implementations, evaluating further comprises: constructing a first probability distribution from the first feature vector and a second probability distribution from the second feature vector. Constructing the first probability distribution may comprise: discretising respective first feature vectors of the plurality of cells of the patient; and constructing a normalised histogram. Constructing the second reference probability distribution comprises: discretising respective second feature vectors of the reference cells; and constructing a normalised histogram.

In some implementations, determining comprises: when the probability distance metric between the plurality of cells of the patient and one of the reference cells, is larger than a predetermined threshold, classifying the cell into the corresponding type of the reference cells.

In some implementations, evaluating further comprises: performing a partitioning analysis on the second feature vector such that a PCA space defined by the principal components is partitioned into a second number of regions.

In some implementations, the partitioning analysis comprises k-means clustering.

In a second aspect of the invention, there is provided a method of classifying a sample of cells of a patient into one or more defined types, comprising:
investigating the sample of cells of the patient using the method of the first aspect of the invention to obtain a sample feature vector (synonymous with the “first feature vector” mentioned above);
providing reference data, wherein the reference data comprises one or more reference feature vectors (synonymous with the “second feature vector” mentioned above) obtained for reference cells of said one or more defined types;
carrying out data analysis, comprising comparing the sample feature vector with said reference feature vector(s), and determining, based on the comparison, whether the sample of cells is classified into one of said defined types, and if so, which of the defined types.

In some implementations, the sample is classified into only one of said defined types. Alternatively, the sample may be classified into several of said defined types, e.g. with an associated probability assigned to each type.

A respective reference feature vector may be provided for each of the defined types. Then, the data analysis may involve comparing the sample feature vector with each of the reference feature vectors, to determine whether the patient is classified into one of the defined types, and if so, which of the defined types. The reference feature vector for reference cells of a defined type may be obtained by investigating a sample of the reference cells in accordance with the method of the first aspect of the invention.

Suitably, the sample of cells of the patient is represented by a single sample feature vector. This may be referred to as a sample fingerprint vector. Similarly, each type of reference cell may be represented by a single reference fingerprint vector. The concept of the fingerprint vector is described in more detail below.

In the data analysis step, determining whether the sample of cells is classified into one of the defined types may comprise evaluating a probability distance metric between the sample feature vector and the reference feature vector(s); and determining whether the sample of cells is classified into one of the defined types, and if so, which type. For instance, if the probability distance metric between the sample feature vector and the reference feature vector is within a predetermined threshold, then the sample of cells may be classified into the defined type corresponding to that reference feature vector.

In some implementations, the data analysis may involve using a classification algorithm obtained through machine learning. The classification algorithm may be configured to determine, based on the sample feature vector, whether the patient is classified into one of the defined types, and if so, which of the defined types.

The classification algorithm may be obtained by applying a machine learning model to a set of training data comprising a set of reference feature vectors for the reference cells of said one or more defined types. Each of the reference feature vectors in the set of training data may be labelled as corresponding to one of the plurality of types of reference cells. Thus, in some implementations, the data analysis may further involve training a machine learning model using the set of training data, to obtain the classification algorithm.

In the second aspect, the reference cells of said one or more defined types may correspond to diseased cells from patients which are confirmed to be responsive to a specific medical treatment. Advantageously, in such instances the classification method can be used as a means to predict the responsiveness of a patient suffering from a disease to a specific medical treatment. In other words, if the sample is classified into the same type as a particular reference cell shown to respond to a particular medical treatment, this can be taken to be indicative that the patient is likely to respond well to receiving the same treatment.

Thus, in a third aspect the present invention provides a method of identifying the suitability of a specific medical treatment for treating a patient suffering from a disease, wherein the method involves:
investigating a sample of cells of the patient using the method of the first aspect of the invention to obtain a sample feature vector;
providing reference data, wherein the reference data comprises one or more reference feature vectors obtained for reference cells, the reference cells corresponding to diseased cells from patients (preferably suffering from the same or similar disease as the patient) which are confirmed to be responsive to the specific medical treatment; and
carrying out data analysis, comprising comparing the sample feature vector with said reference feature vector(s), and determining the similarity of the sample of cells to the reference cells. In such instances, a greater degree of similarity may be indicative of a greater suitability of the specific medical treatment for treating the disease.

In some implementations, the disease is cancer. In such implementations, the specific medical treatment may be, for example, chemotherapy, checkpoint therapy or CAR-T cell therapy.

Optionally, the method may involve identifying a suitable medical treatment for the patient from a range of different specific medical treatments. In such instances, the reference data comprises a plurality of reference feature vectors each relating to reference cells confirmed to be responsive to one of multiple specific medical treatments. For example, the disease may be cancer, and the multiple specific medical treatments may be two or more of chemotherapy, checkpoint therapy or CAR-T cell therapy. In such instances, the data analysis step may comprise determining which of the reference cells the plurality of cells of the patient is most similar to.

In some implementations of the second aspect of the invention, the reference cells of said one or more defined types correspond to therapeutic cells (e.g. CAR-T cells) confirmed to achieve a specific medical outcome.

In a fourth aspect, the invention provides a method of identifying T cells that may be used for CAR-T cell therapy, using the classifying method of the second aspect.

In one implementation, the method involves classifying a sample of T cells based on a comparison with reference cells corresponding to CAR-T cells confirmed to achieve a specific medical outcome. Optionally, the sample of T cells is a sample of CAR-T cells (i.e. after genetic modification).

In this implementation, the method may involve identifying whether a sample of cells from a patient is suitable for use as therapeutic cells in CAR-T cell therapy, comprising:
investigating the sample of cells using a method according to the first aspect of the invention to obtain a sample feature vector;
providing reference data, wherein the reference data comprises one or more reference feature vectors obtained for reference cells, wherein the reference cells are CAR-T cells from patients with a known therapeutic outcome; and
carrying out data analysis, comprising comparing the sample feature vector with said reference feature vector(s), and determining the similarity of the sample of cells to the reference cells. In instances in which the sample vector is determined to be similar to a reference feature vector for reference CAR-T cells known to produce a successful therapeutic outcome, a greater degree of similarity between the sample feature vector and this reference feature vector may be taken to be indicative of a greater suitability of the therapeutic cells for use in CAR-T cell therapy.

In such implementations, the one or more species of proteins detected in the investigation step is, or comprises, CAR.

Alternatively, it is possible to classify a sample of T cells based on a comparison with reference cells corresponding to non-transformed T cells known to be effective for use in CAR-T cell therapy. Specifically, it is believed that the amounts of different types of T cells in a sample can influence the suitability of such cells for use in CAR-T cell therapy. In such instances, the one or more species of proteins detected may correspond to one or more of (i) a surface marker for naïve T cells (ii) a surface marker for memory T cells, (iii) a surface marker for effector T cells (iv) a surface marker for exhausted T-cells.

In this implementation, the method may involve identifying whether a sample of T cells from a patient is suitable for use as therapeutic cells in CAR-T cell therapy, comprising:
investigating the sample of T cells using a method according to the first aspect of the invention to obtain a sample feature vector (preferably wherein the one or more species of proteins detected may correspond to one or more of (i) a surface marker for naïve T cells (ii) a surface marker for memory T cells, (iii) a surface marker for effector T cells (iv) a surface marker for exhausted T-cells);
providing reference data, wherein the reference data comprises one or more reference feature vectors obtained for reference T cells confirmed to be suitable for CAR-T cell therapy; and
carrying out data analysis, comprising comparing the sample feature vector with said reference feature vector(s), and determining the similarity of the sample of cells to the reference cells.

In the third and fourth aspects above, the similarity between sample vectors and reference vectors may be assessed on a probabilistic basis. For example, the similarity may be evaluated based on a probability distance metric (as described above), e.g. with a greater probability distance metric being indicative of greater similarity. Alternatively, the probability may be obtained through a machine learning assessment, e.g. by applying multinomial regression and interpreting the softmax outputs as probabilities. The method may involve applying a threshold criteria to assess suitability.

In a further aspect, the invention provides a method of therapy, comprising identifying a suitable medical treatment for a patient using the third aspect of the invention, and administering said medical treatment to the patient.

For example, the invention may provide a method of treating a patient suffering from cancer, comprising:
investigating a sample of cells (e.g. tumor cells) of the patient using the method of the first aspect of the invention to obtain a sample feature vector;
providing reference data, wherein the reference data comprises at least two reference feature vectors selected from the following categories:
(i) a reference feature vector obtained for reference cells from a patient suffering from the same cancer which are confirmed to be responsive to a chemotherapy;
(ii) a reference feature vector obtained for reference cells from a patient suffering from the same cancer which are confirmed to be responsive to CAR-T cell therapy; or
(iii) a reference feature vector obtained for reference cells from a patient suffering from the same cancer which are confirmed to be responsive to checkpoint therapy;
carrying out data analysis, comprising comparing the sample feature vector with said reference feature vectors and calculating the degree of similarity between the sample vector and each reference feature vector;
selecting a reference feature vector having a degree of similarity satisfying a predetermined criterion (for example, the reference feature vector having the highest degree of similarity to the sample feature vector); and
treating the patient with the same therapy as the selected reference feature vector.

In such aspects, the one or more species of proteins detected in the investigation may be, for example, one or more of CTLA-4, PD-1, PD-L1, CD19, and CSF1R.

In another aspect, the invention provides a method of producing CAR-T cells, comprising identifying a suitable set of sample cells from a patient according to the fourth aspect of the invention, and genetically modifying the sample cells to create CAR-T cells.

In another aspect, the invention provides a method of carrying out CAR-T cell therapy, comprising identifying a suitable set of sample cells according to the fourth aspect of the invention, genetically modifying the sample cells to create CAR-T cells, and administering the CAR-T cells to a patient.

In other words, the invention may provide a method of carrying out CAR-T cell therapy of a patient suffering from cancer, the method comprising:
investigating a sample of candidate CAR-T cells using a method according to the first aspect of the invention to obtain a sample feature vector;
providing reference data, wherein the reference data comprises one or more reference feature vectors obtained for reference cells, wherein the reference cells are CAR-T cells confirmed to show therapeutic benefit against the same cancer; and
carrying out data analysis, comprising comparing the sample feature vector with said reference feature vector(s), and calculating the similarity of the sample of cells to the reference cells, and determining whether the calculated similarity exceeds a pre-defined threshold;
administering the CAR-T cells to the patient if the similarity exceeds said pre-defined threshold.

In a separate aspect, the present invention provides computer-implemented systems configured to carry out the methods of the present invention.

In a separate aspect, the present invention provides a computer processor configured to carry out the methods of the present invention.

### BRIEF DESCRIPTION OF THE DRAWINGS

Certain embodiments of the present invention will now be described, by way of examples, with reference to the accompanying drawings, in which:

FIG. 1 is a flowchart that illustrates a method of detecting one or more molecule species on the surface or within the cells followed by cellular segmentation.

FIG. 2 is a flowchart that illustrates a method of investigating the spatial organization of molecules and the spatial interaction of cells.

FIG. 3 is a flowchart that illustrates a method of classifying patients' cell distributions into one of types of reference cell populations.

FIG. 4a shows an image that illustrates the clusters on a cell defined at various length scales.

FIG. 4b shows a graph that illustrates a HDBSCAN cluster tree.

FIG. 5a is a table which illustrates an example of classification of a test patient's tumor sample based on data obtained from reference patient samples.

FIG. 5b shows exemplary results of the method described herein performed on the data vectors of the three different patients.

FIG. 6a is a table which illustrates an example of the classification of transformed T cells into subpopulations.

FIG. 6b shows the results of a dimension reduction analysis and a partitioning analysis on the data vectors obtained from the CAR-T cells of the test patient.

FIG. 7 is a flowchart that illustrates a method of classifying a cell.

FIG. 8 is a schematic of apparatus suitable for carrying out the methods of the invention.

FIG. 9 is a schematic of localisations of two detected protein markers on a T-cell, showing clustering of the proteins.

### DETAILED DESCRIPTION

The use of cell surface markers forms an increasingly important part of the management of various diseases, for example, in risk assessment, screening, differential diagnosis, prognosis, prediction of response to treatment, and monitoring progress of disease.

Cell therapy is a therapeutic approach comprising the injection, implantation, or other administration of viable cells into a patient. This may involve replacing diseased or dysfunctional cells with healthy, functioning ones. Cell therapy may be applicable to various conditions and diseases, including cancer, neurological diseases such as Parkinson disease and amyotrophic lateral sclerosis, spinal cord injuries, and diabetes.

Immunotherapy is a specific type of cell therapy that is used to treat patients, typically cancer patients, that involves the use of various components of the immune system. Immunotherapeutic approaches generally either improve an immune system response, or initiate one, such as by means of adoptive cell therapies.

An important determinant of the success or failure of all cell therapeutic approaches is the interaction of the administered cells with the cells of the recipient patient, mediated by signalling molecules on the surface of one or both of these populations of cells. The present disclosure provides methods to quantify and categorise the spatial distribution of signalling molecules mediating cell-cell interactions at a specific time point. In particular, the present disclosure provides methods to analyse and categorise the interaction of cancer cells with potential immunotherapeutics such as adoptive cell therapeutics.

A novel algorithm is described, called “Outcome PRediction Algorithm” (OPRA) for predicting outcomes of cell-mediated therapies, such as, for example, involving engineered or native immune cells, checkpoint inhibitors or other therapeutics. Predicting the reaction between cells, such as immune cells and tumor cells in both solid and liquid tumors, is a precursor to predicting treatment outcome. It has been found that the interaction between these cells can be predicted by characterizing the spatial distribution of individual surface proteins on the surface of target and effector cells, such as individual protein antigens and immuno-modulatory molecules on single tumor and immune cells. The analysis of an even higher level spatial organization is achievable in case of solid tumors or tissues where the spatial distribution of the analysed cells contains additional information which is taken into consideration.

It has been found that using the disclosed methods, the spatial distribution of these molecules and cells can be determined at all length scales. The method takes into consideration all spatial organizations, from individual molecules to clusters of molecules, to clusters of clusters including the cell-cell interaction and spatial heterogeneity levels.

As an example, single-molecule localization microscopy may be used, together with an algorithm called Hierarchical Density-Based Spatial Clustering of Applications with Noise (HDBSCAN), to quantify the multilevel spatial organization of the molecules of interest in the cell. The coordinates of the molecules may be derived by localizing fluorophores with which the molecules are tagged.

The features that define the spatial organisation of the molecules on the cell surface may then be correlated with specific properties of the target-effector cell interaction, such as an immune cell-cancer interaction. For example, the spatial organisation of surface receptors on chimeric antigen receptor (CAR) T-cells may be used to predict the ability of the cells to specifically and efficiently neutralise particular cancer cells, or to predict undesired behaviours of the cells, such as off-target activity.

Likewise, the arrangement of receptors on the surface of tumor cells and the spatial distribution or interaction of the detected cell types in case of solid tumors may be used to predict the likelihood of successful elimination of the tumor by immunotherapy.

Thus, the disclosed methods, which provide unprecedented information about the cells based on the spatial organization of molecules of a particular potentially therapeutic cell or an individual cancer cell from a specific patient, will lead to various advantages, including precision medicine and more accurate selection of the exact type of therapy and dosage to be administered to a specific patient.

The disclosed approach thus provides a method comprising super-resolution microscopy based analysis as a companion or complementary diagnostics tool which can be applied to all types of cell therapies, and in particular immunotherapies, and to various types of disease.

Quantifying the organization of molecules in therapeutic cells, such as immune cells for use in adoptive cell transfer therapies, can further be used to refine the development and manufacturing of the cell-based therapies. For example, in novel cell-based immunotherapies, membrane receptors on immune cells are genetically engineered to target cancer cells more efficiently (for example, CAR-T cells). It has been found that the spatial organization of engineered surface receptors on immune cells can be correlated with the efficacy and side effects of the therapeutic cell product.

Method

In relation to the treatment of cancer as an example, the detection and quantification of the absolute levels of expression of various biomarkers on cancerous cells and tumors is currently used in clinics as a method for tumor diagnosis and patient stratification. Determining the level of expression of biomarkers is performed, for example, by immunochemical methods in combination with flow cytometry, fluorescence or non-fluorescence microscopy.

More recently a number of methods have been developed which rely on multiplex analysis of tumor proteome or transcriptomes. Although these methods are widespread they have a number of shortcomings, including, for example: insufficient sensitivity to detect low copy numbers (levels); the inability to provide in-depth information about cellular or subcellular localisation or organization; and/or the inability to provide information related to spatial context. These shortcomings may result in incomplete or even incorrect patient stratification and inclusion for a particular therapy.

The disclosed method comprises the use of single-molecule super-resolution fluorescence microscopy with machine learning algorithms to quantify and categorise the spatial distribution of cell surface molecules to classify cells and cell populations in a sample, for example to predict properties of the sample based on a comparison to a reference sample.

Single-cell sequencing and spatial transcriptomics yields gene expression data that has brought about a new understanding of the distribution of individual cell types in populations which were previously assumed to be homogeneous. The high dimensional gene expression data is often projected to lower dimensions with algorithms such as Principal Component Analysis (PCA), T-distributed Stochastic Neighbor Embedding (t-SNE), or Uniform Manifold Approximation and Projection (UMAP). Cell types may subsequently be distinguished via clustering algorithms run on the lower dimensional data, such as the k-means clustering or graph based methods. The distribution of cell types yields novel information that is currently the subject of many research publications, and it holds great promise for future use in clinical workflows.

In view of existing knowledge of gene expression cell heterogeneity, especially in tumors, there is a need to consider previously unobtainable data based on protein and cell distributions detected with higher sensitivity when attempting to understand tumor pathologies. Gene expression as measured by mRNA profiling is only indirectly correlated to protein levels at their target location. Especially in tumor cells, transport of receptor proteins and insertion, or translation of proteins directly into membranes, can be disturbed. A direct measure of the quantities of a specific protein in a particular location in which the protein performs its function is therefore crucial to understanding protein heterogeneity.

Beyond copy numbers, the spatial distribution or organisation of proteins can have a great impact on their function. For example, in the case of immune receptors, it is known that the density of receptors on the surface of the cell can modulate the response. The outcome of treatment may also be influenced by the way the cell types are organised and interact with each other within tissues. Therefore, under optimal circumstances the following criteria/features would need to be measured to fully quantify and assess protein spatial distribution:
1. protein copy numbers
2. spatial distribution of proteins at any given time
3. trajectory of the motion of the proteins as a function of time, particularly in a scenario where the cell engages in an immune interaction with another cell.
4. cellular heterogeneity and cell-cell interactions.

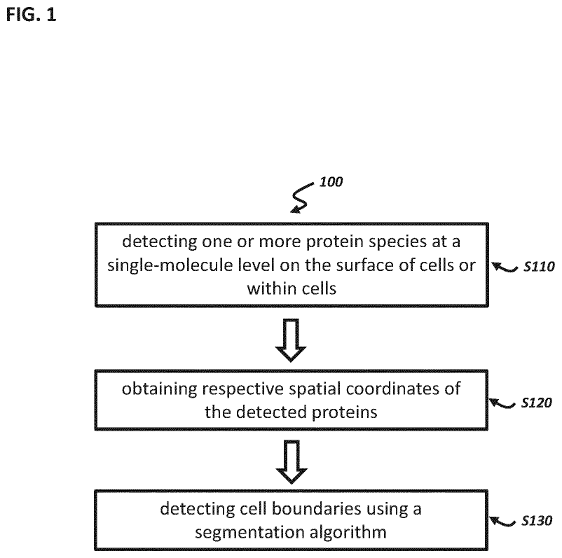

FIG. 1 Is a flowchart that illustrates a method of detecting one or more molecule species on the surface or within the cells followed by cellular segmentation.

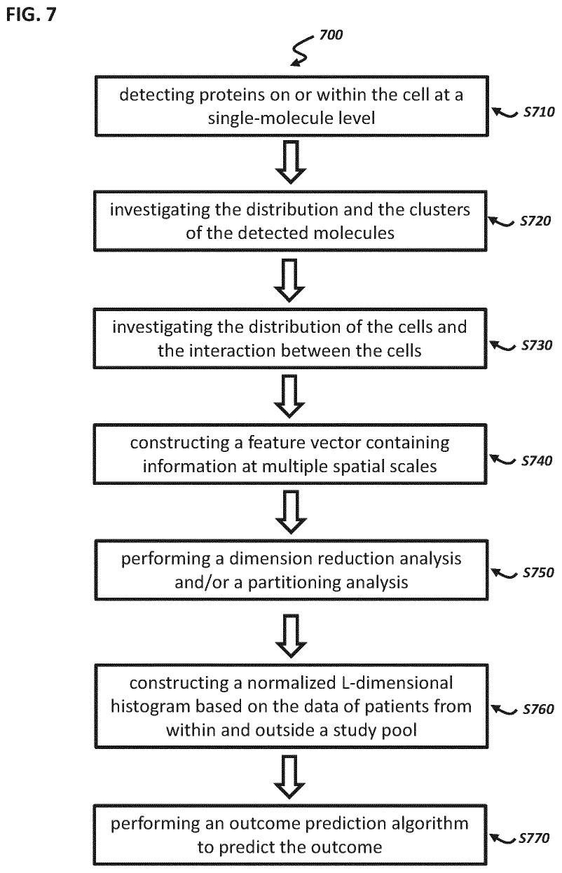

The method 100, which corresponds to a detailed description of step 710 of FIG. 7, relates to detecting one or more molecule species on the surface or within the cells followed by cellular segmentation. In particular, the method 100 relates to detecting proteins (and other biomolecules) and their spatial coordinates on the surface or within individual cells delineated by image segmentation.

At step 110, one or more species of proteins are detected at a single-molecule level on the surface of the cells or within cells.

In some implementations, fluorescence microscopy techniques may be used to detect individual molecules and map their spatial coordinates in a cell. For example, direct Stochastic Optical Reconstruction Microscopy (dSTORM) can be used to detect proteins on or within a cell. The method can be performed on any cell type, and as an example, the method has been established using immortalized human T cells (Jurkat cells). Likewise, the method can be performed using any relevant protein, and as an example, the beta subunit of the T cell receptor may be used.

To facilitate fluorescence microscopy, the proteins of interest can be labelled with fluorescent markers comprising a fluorophore, such as a fluorescent dye, quantum dot or fluorescent protein. To target the fluorescent marker to the protein of interest, the fluorescent marker may be specific to the protein of interest. For example, the fluorescent marker may be or comprise a capture molecule labelled with a fluorophore. The capture molecule may be, for example, an antibody, aptamer, nucleic acid, polypeptide, or a purified or synthetic ligand.

In such implementations, the method of investigating the plurality of cells comprises a labelling step, prior to said detecting one or more species of proteins on each of the plurality of cells. The labelling step may involve incubating the cells with a fluorescent marker specific to the protein of interest. Alternatively, the labelling step may involve modifying the cells so as to express the protein of interest labelled with a fluorescent protein. In such implementations, the step of detecting one or more species of proteins on each of the plurality of cells and obtaining respective spatial coordinates consists or comprises of carrying out single molecule localisation microscopy, for example using dSTORM or fPALM.

In some implementations, only one type of protein may be detected for the analysis or the investigation.

In some implementations, two or more types or species of proteins (and/or further biomolecules) may be detected for the analysis or the investigation. In this case, each species may be labelled with a distinct fluorescent marker such that each species can be differentiated (e.g. through being detected in different colour channels).

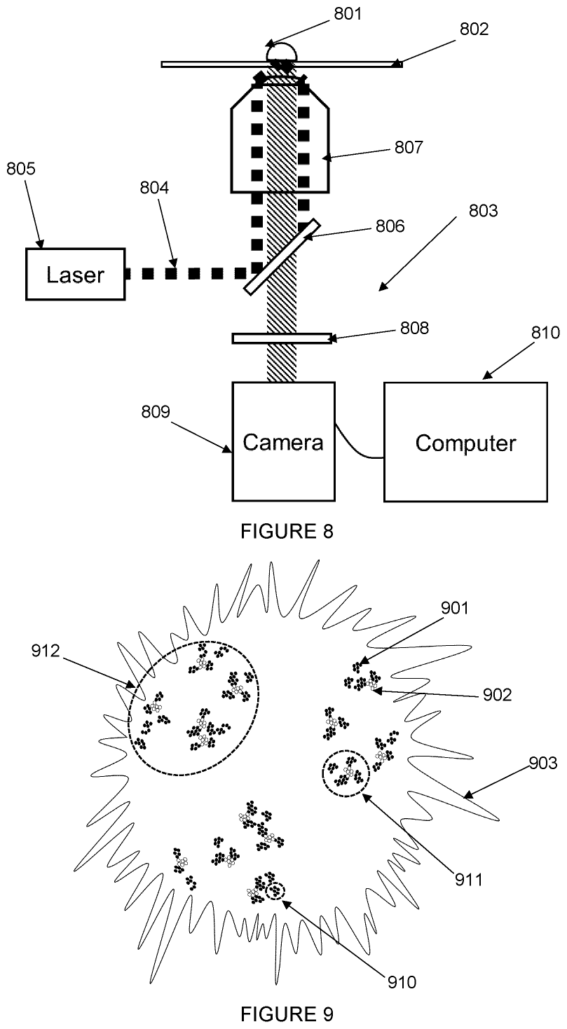

A super-resolution fluorescence microscopy system suitable for carrying out step 110 is shown in FIG. 8. FIG. 8 shows a sample 801 mounted on coverslip 802. The sample 801 contains a plurality of tumor cells from a patient suffering from cancer, which are immobilised on the coverslip 802 and immersed in an imaging buffer. The imaging buffer is compatible with dSTORM, containing a reducing agent (e.g. a primary thiol such as β-mercaptoethanol (BME), mercaptoethylamine (MEA), dithiothreitol (DTT) or L-glutathione) and an oxygen scavenging system (e.g. the combination of glucose oxidase and catalase, or the combination of protocatechuic acid (PCA) and protocatechuic dioxygenase (PCD)). The cells have been labelled with a dSTORM compatible fluorescent probe having specificity to a protein expressed on the cell surface, and have been fixed prior to imaging to preserve clustering information. The dSTORM compatible fluorescent probe includes a photoswitchable fluorophore, which is able to switch from a dark state to an emissive state.

The sample 801 is interrogated by Total Internal Reflection Fluorescence Microscopy (TIRFM) system 803. In the TIRFM system 803, excitation beam 804 from laser 805 is reflected by dichroic mirror 806 so as to pass through the edge of objective lens 807, and totally internally reflect off the top surface of coverslip 802. This creates an evanescent field, which switches a small proportion of the photoswitchable fluorescent probes from a dark to an emissive state. Fluorescence emission from the emissive fluorescent probes is then collected by objective lens 807 and passes through dichroic mirror 806 and optical filter 808 before being detected on EMCCD camera 809. Signal from the emissive fluorescent probes then disappears, either due to the fluorophore switching back to a dark state or photobleaching. Through control of conditions (in particular laser power), the density of photoactivated fluorescent markers in each image recorded by the camera is such as to allow individual fluorescent markers to be identified as separate points. By acquiring multiple images, it is possible to gradually construct an image of individual fluorescent markers across the cell surface.

In addition to acquiring fluorescence data, TIRFM system 803 also acquires a white light image of each interrogated cell, which can be mapped onto the fluorescence data.

Data from EMCCD is fed to computer 810 for storage and processing. Computer 810 is configured to carry out steps 120 and 130 depicted in FIG. 1.

At step 120, respective spatial coordinates of the detected single molecules in the field of view containing the cells are obtained using a single molecule localization algorithm. This step corresponds to a detailed description of step 710 of FIG. 7.

In some implementations, a super-resolution microscopy technique which can achieve a spatial resolution of 10 nm to 20 nm may be suitable for counting individual proteins and measuring the hierarchical organization of proteins forming structures like clusters, and clusters of clusters, etc. allowing detection of changes or differences in organisation which would otherwise go undetected.

However, the method provided herein is not limited to fluorescence microscopy techniques or super-resolution microscopy techniques. Any techniques, including non-optical techniques, capable of counting and localising individual proteins with a resolution required for identifying the organization of proteins on the surface or within cells may be used.

In some implementations, direct Stochastic Optical Reconstruction Microscopy (dSTORM), a super-resolution microscopy technique, may be used for the detection of individual molecules and mapping of their coordinates in a cell.

The data obtained with the dSTORM technique is a continuous coordinate space map with the locations of fluorophore-tagged proteins or molecules of interest.

The term “localization” in this specification refers to an act of estimation of the location of a molecule, protein or a fluorophore or the estimated spatial location estimated therefrom.

A schematic showing the localization of molecules on a cell surface is shown in FIG. 9. FIG. 9 shows two types of fluorescent markers, each having specificity to different surface proteins, one marker represented by black circles 901 and the other by white circles 902. In this case, the fluorescent signal from the markers has been fitted with a 2D Gaussian function, and the circles are centred at the peak of each Gaussian with the circle radius corresponding to the standard deviation of the fit (generally taken to be a measure of the localization accuracy). For ease of understanding, the fluorescence data is overlaid with a white light image of the cell 903. From a qualitative assessment, it can be seen that black circles 901 group into clusters 910, which group into larger clusters 911. These larger clusters 911 themselves cluster into larger regions 912. In other words, clustering behaviour is seen across multiple length scales. Moreover, it can be seen that white circles 902 form small clusters 920 which appears to show the same clustering behaviour as black circles 901. The method of the invention goes beyond this qualitative assessment, and allows the characteristics of the clustering behaviour across different length scales to be quantitated and utilised to inform treatment decisions. For the avoidance of doubt, the skilled reader will recognise that FIG. 9 is included for illustrative purposes only, and is not intended to be to scale.

Moving on to step 130, cell boundaries are detected using a segmentation algorithm. The segmentation algorithm is applied which allows the delineation of cellular boundaries in both tissue samples and isolated cells. The segmentation algorithm allows the detection of cellular boundaries. For example, the segmentation algorithm can be applied to fluorescence images and/or brightfield images of the cell. Then the segmentation area is applied to the single molecule localization data as a mask. Localizations of which coordinates fall on the border or within the mask are then assigned to that particular cell. Each mask corresponding to a single cell is then given an identifier which will be used in the analysis of cell-cell interactions. This step corresponds to a detailed description of step 720 of FIG. 7.

For example, after the molecules of interest are detected and localised to yield molecular coordinates using a suitable detection technique such as dSTORM (steps 110 and 120) and after the cellular boundaries are identified (step 130), a spatial distribution analysis algorithm, such as HDBSCAN analysis, is applied to the molecular coordinates to identify clustering of the spatial coordinates (step 210).

Subsequently, in some implementations, principal component analysis and k-means clustering may be further applied to the result of the spatial distribution analysis algorithm. This will be discussed in more detail later.

The method provided herein may be used for application such as patient stratification and quality assessment of cell therapy products, a reference library of patient data is assembled by applying the method to data obtained from the cells of the patient. For example, this may uniquely characterise patient tumor samples, tumor neutralizing potential of native T-cell populations in the presence of drug molecules, and therapeutic immune cells.

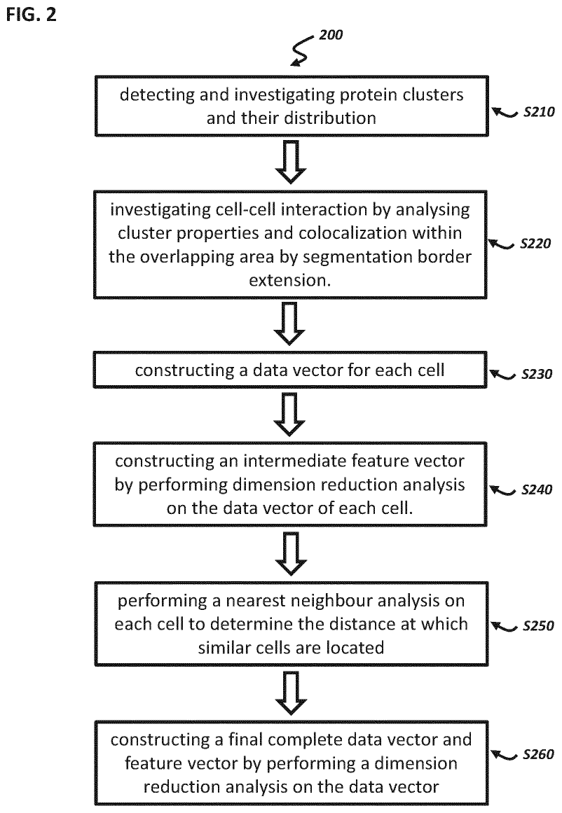

FIG. 2 is a flowchart which illustrates a detailed method of investigating the spatial organization of molecules and the spatial interaction of cells.

In particular, the method 200 corresponds to the detailed steps of steps 720 and 730 of FIG. 7, which is characterising a spatial organisation of proteins and the spatial interaction of cells.

The method 200 relates to the analysis of the distribution (Category 1) and clustering (Category 2) of the detected molecules in each cell (step 210) and to the analysis of cell-cell interactions (Category 3) (step 220) and construction of data vectors and feature vectors (steps 230, 240, 250).

In step 210, protein clusters and their distribution are detected and investigated. The distribution and clustering of the localized molecules are evaluated. Clusters are detected using algorithms such as HDBSCAN and evaluation is performed using the algorithms detailed below and the output values are then used for the construction of data vectors for each cell.

Category 1. Localization Distribution
1.a. Number and density of localizations for each type of molecule or protein
1.b. Distance between localizations across multiple types of molecules or proteins (nearest neighbour analysis): the average distance between the localizations of one channel to the neighboring localizations of the other channel. This is a very basic form of estimating whether the there is some colocalization tendency.
1.c. Ripley's K function: Ripley's K function can be used to assess the distance at which most clusters can be observed.

Category 2. Cluster Level

The clusters can be obtained from a spatial distribution analysis algorithm, which will be explained in more detail later.
2.a Mean, standard deviation and median cluster radius/diameter at multiple length scales.
2.b Mean, standard deviation and median cluster area at multiple length scales.
2.c Mean, standard deviation and median cluster density at multiple length scales.
2.d Mean, standard deviation cluster shape at multiple length scales. The shape of a cluster can be described by a value obtained from dividing the value of the major axis by the value of the minor axis. This approximates circularity of a cluster for example.
2.e Mean, standard deviation of number of localizations per cluster at multiple length scales.
2.f Mean absolute deviation of number of localizations per cluster at multiple length scales. The mean absolute deviation is a way to describe the variability of the number of localizations which make up the clusters at a specific length scale. For example, at small length scale such as 50 nm, the variability in terms of localizations/cluster is low (10-100 localizations per cluster for example). At higher length scales, where cluster sizes become more heterogeneous, the number of localizations per cluster becomes heterogeneous as well. For example, some clusters may have 100 localizations while others will have more than 10000. Therefore, the mean absolute deviation will also increase. Thus, the aim of this analysis is to give an additional value (parameter) describing the heterogeneity of the sample at each analysed length scale of the cluster hierarchical tree.
2.g Maximum absolute deviation of number of localizations per cluster at multiple length scales.
2.h Mean number of clusters at multiple length scales.
2.i Mean number of clusters within ranges (bins) defined by at least 2 length scales (i.e. number of clusters between the 50 and 100 nm length scale interval).
2.j Median number of localizations per cluster at the mentioned length scales.
2.k Median absolute deviation of number of localizations per cluster at multiple length scales.
2.l Mean absolute difference between the values of a given feature (i.e number of localizations per cluster at each length scale) obtained through multiple length scale analysis of the spatial distribution analysis algorithm.
2.m Ratio of total number of localizations per cell compared to the number of localizations in clusters at each length scale.
2.n Mean number of nanodomains (subclusters) per cluster (HDBSCAN and SR-Tesseler).
2.o Subclassification of colocalized cluster populations based on cluster features (colocalizing cluster size, density, shape, number of localizations and nanodomains, number of clusters of each analysed molecule species per colocalization area (cluster composition). Colocalization refers to the coexistence of the molecules (e.g. proteins) of interest within a defined area. Subclassification refers to the possibility that the colocalizing clusters show some common traits which differentiates them from the clusters that do not colocalize. These traits allow further classification of clusters within cells. For example, 50 nm diameter clusters colocalize with the clusters from the other channel while smaller or bigger clusters show no colocalization. Diameter can be changed to the other descriptors mentioned. Algorithms such as SODA (Statistical Object Distance Analysis) can be used to obtain the cluster colocalization data needed to perform these analyses.
2.p Degree of colocalization (i.e. ratio between total number of detected clusters for each molecule species per cell vs. the number of colocalizing clusters; the number of molecule species considered for colocalization is equal to or greater than 2: three-way colocalization). This allows the analysis of the proportion of clusters out of the total number of clusters (for a protein) which fall within a distance (which we consider colocalization distance) from a cluster of another protein. e.g. out of 1000 clusters of protein A, 800 clusters are within the “colocalization distance” of clusters from protein B. This includes preferential colocalization in case of three or more molecule species; expressed as the percentage or number of clusters colocalizing with clusters of one or the other molecule species out of the total number of clusters or total number of colocalizing clusters for a specific molecule species. Colocalization algorithms used to obtain the above-mentioned values may include methods such as SODA.
2.q Mean, median, standard deviation of distance between clusters of the two detected proteins.
2.r Cluster stability at different length scales. Cluster stability is a parameter which shows whether a cluster persists over multiple rounds of clustering or not at a specific length scale.
2.s Average distance of clusters compared to the center of mass of the measured
2.t Cell symmetry (symmetry index calculated based on the distribution of clusters).
2.u Colocalization distances between clusters in overlapping areas, where the colocalization distance is defined as the distance between clusters of two different molecules (e.g. proteins) cluster species which coexist (interact) within a defined maximum radius. Beyond this maximum defined radius, colocalization values are considered biologically irrelevant/not colocalizing/interacting. Colocalization distance refers to the distance between clusters of two different protein species which coexist within a defined maximum search area (radius). The data for 2.0 and 2.v are obtained from performing colocalization analysis (e.g. using SODA) on the clusters which are located within the area obtained by extending the cellular segmentation area (used for detecting cell-cell interaction described below).
2.v Number, area, density, shape and number of localizations of clusters which fall within overlapping areas.

To obtain the data vectors of a fixed length, the spatial map obtained with the dSTORM technique, which includes the localizations, may be processed by applying a spatial distribution analysis algorithm or a spatial clustering analysis algorithm.

In some implementations, the spatial distribution analysis algorithm may include applying radial distribution functions evaluated at a fixed set of radii. However, the distribution function does not directly yield any information on copy numbers (for criterion 1 discussed above) which must be obtained differently.

In some implementations, the spatial distribution analysis algorithm comprises Hierarchical Density-Based Spatial Clustering of Applications with Noise (HDBSCAN).

In some implementations, the spatial distribution analysis comprises algorithms such as SR-Tesseler.

In some implementations, to obtain copy numbers as part of the data vector, a hierarchical clustering algorithm may be used that can detect individual proteins at the lowest spatial hierarchy.

In some implementations, to describe the spatial distribution of proteins at any given time point (criterion 2), the data from the higher spatial scales of the hierarchy tree obtained using the spatial distribution analysis algorithm such as HDBSCAN or non-hierarchical algorithm such as SR-Tesseler.

In this specification, the term “cluster” refers to a collection or a group of points, which are closely packed together with a larger number of nearby neighbours than the overall distribution such that the density within the cluster is above that of the random distribution. The points can be the spatial coordinate of the proteins or a data point on a parameter space such as reduced dimensional space in the Principal Component Analysis.

Subsequently, in some implementations, principal component analysis and k-means clustering may be further applied to the result of evaluations of categories 1 and 2 following the spatial distribution analysis algorithm. This will be discussed in more detail later.

At step 220, cell-cell interactions are investigated by analysing cluster properties and colocalization within the overlapping area by segmentation border extension. This step corresponds to the detailed description of steps 720 and 730 of FIG. 7. As specified in step 130, each cell is segmented and all molecules and their clusters which fall within this area are assigned to the respective cell. The segmentation area may be extended in all directions for each cell by the minimum of 10 nm (the measured distance between each side of the immunological synaptic cleft), but not higher than 1000 nm, such that it is ensured that the space between the original segmentation border and the extended border will contain molecule species and their clusters (clusters of minimum 2 different proteins for example (PD1-PDL1)) from both cells. This is the definition of ‘overlapping areas’. The minimum segmentation border extension distance is defined based on a biologically relevant value. Approximately 10 nm would be the minimum distance across the cytoplasmic facing side of the two cells participating in an immunological synapse. A subsequent colocalization step (using an algorithm such as SODA) is then applied which allows the measurement of distances between the clusters of the two molecules of interest. The colocalization distance value is then added as a feature for each cell. In addition to the colocalization measurements, the number, area, density, shape and number of localizations of clusters which fall within the overlapping area is also calculated and added to the features or the feature list as listed in category 2 (features of the clusters within the overlapping area obtained at a single user defined lengthscale). When cell-cell interactions are considered, cluster colocalization values are investigated. Therefore, the output values are part of category 2—specifically 2.0 and 2v. Practically, these output values are compiled in a document such as a .csv file, which can then be used for the generation of a data vector. In this way, a shift in any of the values described above may indicate a physiologically relevant interaction between two or more adjacent cells which may be specific for a certain cancer phenotype.

Category 3.
3.a Cell neighbourhood component quantification: median, mean, standard deviation of distance between cells (Cell neighbourhood component quantification obtained through nearest neighbour analysis for each cell within a set maximum radius. i.e. the distance at which a cell with similar features can be found measured for each cell).
3.b Cell type distribution in relation to a reference cell, a known or defined cell type, defined by a known marker: median, mean, standard deviation of distance between cells. (identified feature or specific marker i.e. CD4). The features 3.a and 3.b will allow the user to estimate the heterogeneity of the samples both locally and at greater distances. A low value indicates that similar cells can be found near. Furthermore, this indicates that similar cells form relatively homogeneous spatial clusters. A higher value indicates that similar cells are dispersed therefore indicating a heterogeneous tissue. Furthermore, neighborhood components of a known cell type will show whether there is a specific distribution of cells around that cell type, and/or is the known cell type evenly distributed or forms spatially defined clusters.
3.c Neighbouring cell cluster colocalisation
3.d Distribution of cells (Ripley's K function): Ripley's K function can be used to assess the distance at which most cell clusters can be observed (including whether the cells are clustered or randomly distributed).

Our analytical pipeline obtained by applying the method described herein may overcome the limitation of detecting only the expression levels and increases the depth of analysis, taking into consideration multiple parameters (Categories 1, 2, 3) which uniquely define the spatial organization and relationships of molecules in cells and interaction between cells. This may be advantageous for immunotherapy.

At step 230, a data vector is constructed for each cell. The parameters used to construct the data vector may include ones belonging to categories 1-2, which uniquely define the spatial organization and relationships of molecules in cells. The features belonging to category 3 describe the distribution and interactions of cells within tissues and contribute to the construction of a feature vector. This step corresponds to a detailed description of step 730 of FIG. 7. To construct the data vector, values from both categories 1 and 2 can be used. In order to assess cell-cell interactions and heterogeneity (such as nearest neighbours) (category 3) first the values for category 1 and 2 are obtained and an intermediate data and feature vector for each cell can be constructed. The dimension of the feature vector can be determined by selecting features. Alternatively, all features can be used and principal component analysis can be applied to assess which features are relevant and have potential biological relevance, to finally determine the dimension of the feature vector.

At step 240, a dimension reduction analysis is performed on the data vector in order to construct an intermediate feature vector for each cell. Any suitable dimension reduction methodology may be used, such as Principal Component Analysis (PCA), t-distributed Stochastic Neighbour Embedding (t-SNE), Uniform Manifold Approximation and Projection (UMAP). For the purposes of this discussion, we exemplify dimension reduction analysis based on Principal Component Analysis (PCA) [the details of which will be described in detail in step 260]. This step corresponds to a detailed description of step 730 of FIG. 7. In step 240, the intermediate feature vector is generated for each cell needed for the nearest neighbor analysis for each cell. Principal component analysis is performed on the intermediate feature vector and the most significant components are stored as input for the nearest neighbor analysis for each cell. The principal component analysis steps are similar to or the same as the procedure in the beginning of step 260.

At step 250 a nearest neighbor analysis is performed for each cell to determine the distance at which similar cells are located. The analysis relies on the intermediate feature vector which contains a set of features describing the cell. Nearest neighbor analysis uses these features describing each cell to calculate the distance at which similar cells can be found. For each cell a radius can be defined to limit the analysis to a maximum defined distance. The nearest neighbour distance between any given cell and its nearest neighbor describes whether similar cells form spatial clusters or are distributed throughout the sample. The dimensions for generating the data vector based on which the intermediate feature vector is obtained (which forms the basis for the nearest neighbour analysis) can be defined using PCA or can be selected manually. The output values are added to the features (category 3) for each cell. This step corresponds to a detailed description of step 730 of FIG. 7. This works as a feedback loop.

After a data vector is generated based on categories 1 and 2, there is a branching point where the data vector is essentially duplicated. One is kept unchanged, which is the data vector carried until step 240. The duplicate will be used for dimension reduction analysis (in this case principal component analysis) in step 240 to generate the feature vector necessary to do the nearest neighbor analysis in step 250. The values from the nearest neighbor analysis are then fed into the original data vector to generate the final complete data vector. The results of the nearest neighbor analysis are added to the data vector carried until step 240, which after the addition of features from category 3 becomes the final complete data vector (step 260).

This allows the detection and quantification of the highest level spatial organization at the cell-cell interaction level. The additional features (dimensions) from the highest spatial scale analysis of each cell are then added to the final complete data vector which will be used for construction of the final complete feature vector used for downstream analysis (step 260).

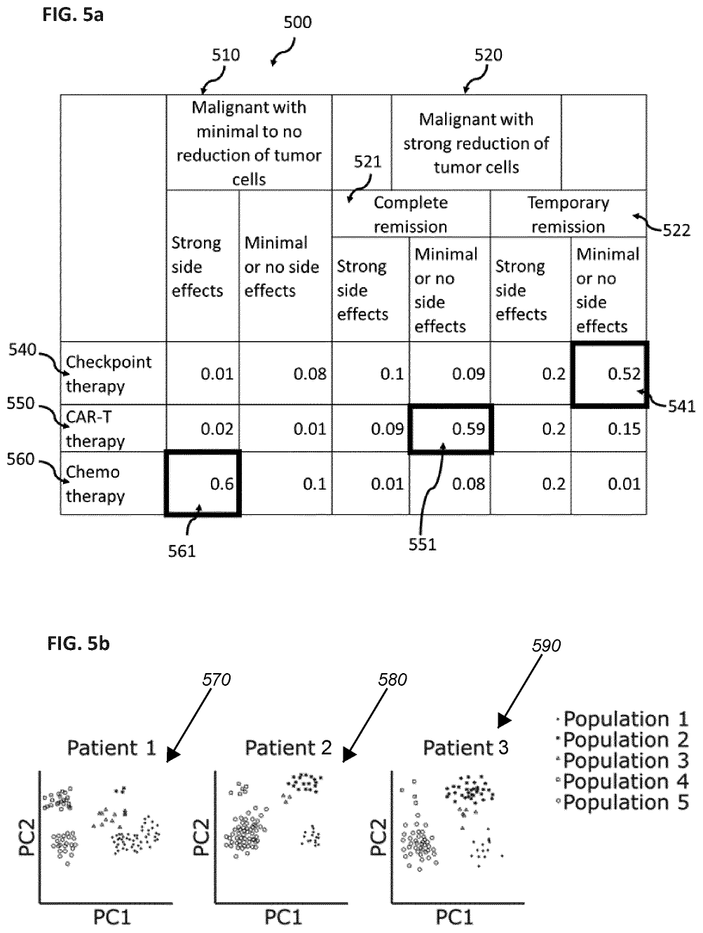

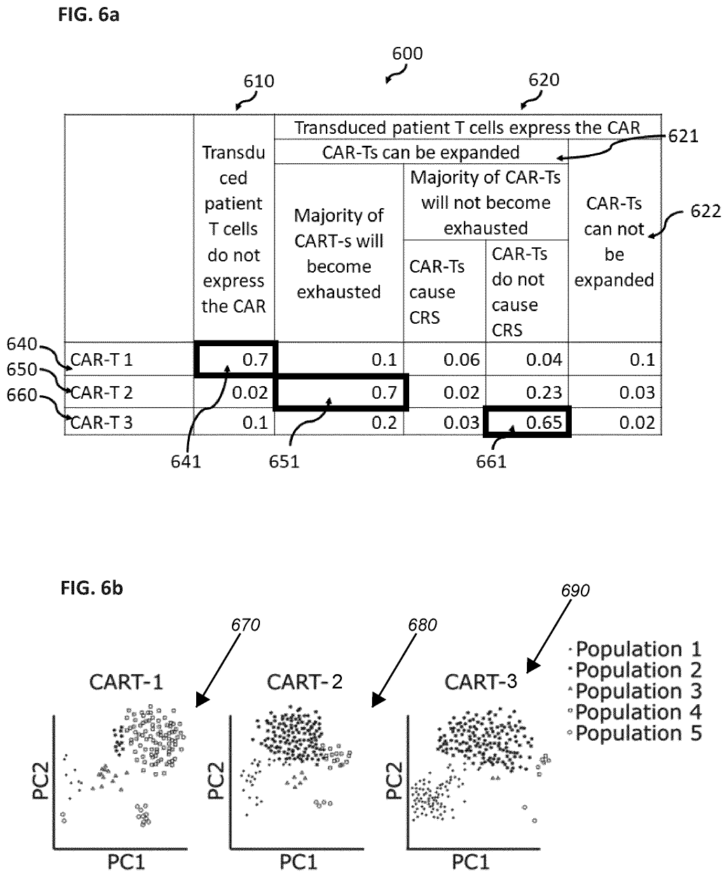

To obtain the fixed size data vector (a final complete data vector) from this hierarchical clustering data, we evaluate a fixed set of properties at a fixed set of spatial scales. For example, properties can be 1. number of clusters, 2. mean, median, SD of area of clusters, 3. mean, median, SD of distance between clusters, 4. mean, median, SD of number of localizations per cluster, at spatial scales 1. 10 nm, 50 nm, 100 nm, 500 nm, 1000 nm. This choice yields a 50 dimensional data vector for each cell. The dimension M of the data vector can be increased to arbitrarily high numbers by choosing more spatial scales, or by including further statistical descriptors or features. This data vector was used in the examples where the detected molecular signatures of the analysis according to the method form the basis of the classification of patient samples shown in FIG. 5 and the classification of CAR-T cells shown in FIG. 6.

Taking into account these parameters the technique may allow a user to perform an in depth single-molecule based cell classification by detecting and quantifying molecular signatures according to protein levels and their spatial organization while taking into account the spatial distribution and interaction of the cells themselves in case of tissues.

At step 260 a final complete feature vector is then constructed by performing a dimension reduction analysis on the final complete data vector. This step corresponds to a detailed description of steps 740 to 760 of FIG. 7.

A final complete data vector is constructed and a final complete feature vector is constructed by performing a dimension reduction analysis on the final complete data vector.

As noted above, the dimension reduction analysis applied in step 240 or 260 may be any suitable technique including, for example, PCA, t-SNE or UMAP. In some implementations, the same dimension reduction analysis technique is applied in each step. In other implementations, different dimension reduction analysis techniques are applied in step 240 compared to step 260.

In some implementations, the dimension reduction analysis comprises a Principal Component Analysis.

The number of L most significant principal components of the final complete data vector (step 260) are kept for the downstream process which can vary between L=2 and L=M, the total length of the data vector. The selected L principal components are then stored.

In some implementations, any further data vectors will not require the PCA algorithm, but can be directly transformed into “feature vectors” in the selected L-dimensional PCA subspace by matrix multiplication.

In some implementations, when cells from a plurality of patients are assessed, there may be two alternative implementations to perform the dimension reduction analysis on the data vector.

In a first alternative implementation, (final complete) data vectors obtained as a result of step 250 may be aggregated from, for example, tumor cells across study patients with the same disease, ideally those patients who have the same disease mechanism. This implementation assumes that data vectors from different patients can indeed be compared, and that patient to patient variation of the data vector for any particular cell type is at a moderate level. In this case, a global dimension reduction analysis can be performed, e.g. PCA to obtain a set of principal components.

In a second alternative implementation, the case is considered where there is a considerable patient-to-patient variation in the data vector for the same cell type (although the number of all cell types might be the same). In this case, a new dimension reduction analysis is performed on each patient, e.g. PCA is performed on each patient without storing any of the principal components for downstream analysis.

Whether alternative implementation 1 or 2 is used in the diagnostic workflow depends on the protein(s) and the disease of interest and which operations are required to create final complete feature vectors which are consistent between patients with consistent cell type populations and statistical analysis of sample variability based on obtained features. The fingerprint vectors are constructed based on the final complete feature vectors.

In some implementations, to make the final complete feature vector more comparable between patients, a partitioning analysis, (a further spatial clustering step on the feature vector) in PCA space may be performed.

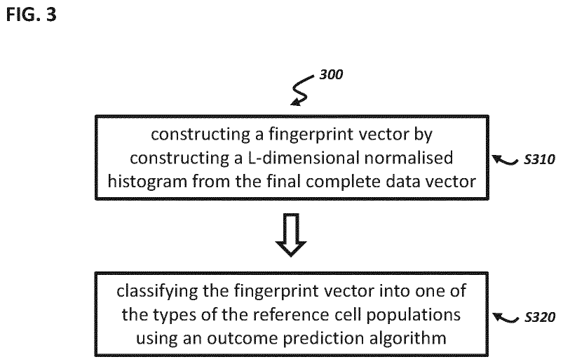

FIG. 3 is a flowchart that illustrates a method of classifying patients' cell distributions 35 into one of the types of reference cell populations.

The method 300 relates to the generation of the fingerprint vector based on which the Outcome PRediction Algorithm (OPRA) can be implemented. The method 300 corresponds to a detailed description of step 770 of FIG. 7.

In some implementations, the patient cell spatial organisation and the reference spatial organisation are characterised by a fingerprint vector of the cell and respective fingerprint vectors of the reference cells.

At step 310 the fingerprint vectors are generated by constructing an L-dimensional normalised histogram from the final complete data vector.

The patients' cells can be classified according to a proximity metric which evaluates similarity between the fingerprint vector of the patient and reference probability distributions of respective reference patient groups.

In some implementations, the L-dimensional space in which the feature vectors of the patient cell and the reference cells are defined, can be discretized over a fixed region that covers the L-dimensional hyperrectangle within which the data points are distributed. A normalized L-dimensional histogram can be calculated by counting the number of data points in each L-dimensional unit block. This histogram is an approximation of the continuous probability distribution of cells in this L-dimensional subspace. This is in this specification defined as the fingerprint vector.

To make the feature vector more comparable between patients, a partitioning analysis, a further spatial clustering step on the feature vector, in PCA space may be performed.

In some implementations, the partitioning analysis comprises k-means clustering. By applying k-means clustering with K clusters, the L-dimensional PCA space may be split into K regions corresponding to K different cell types.

In particular, in the second alternative discussed in step 260, since the L′ most significant principal components will be different from patient to patient, a further clustering algorithm such as k-means clustering may be performed in this case to obtain a feature vector that can be compared between patients. For instance, k-means clustering with K′ number of clusters can be used to partition the L′-dimensional space into K′ regions corresponding to K′ different cell types.

For the reference cells, a patient pool may be provided where the outcome is known after treatment with a specific therapy. The methods 100 and 200 are applied to each of the cells of the patient pool. The reference fingerprint vectors are generated based on the feature vectors from the cells of all patients who received the same therapy and had the same outcome in method 1 and obtain for each M-dimensional data vector (data vector), one L-dimensional feature vector in the L-dimensional subspace (feature vector).

In some implementations, the spatial organisation of patient and reference cells are characterised by the final feature vector of the patient cells and the respective feature vectors of the reference cells.

The generation of a histogram based on patient data is the basis for finding the patterns specific for the respective patient group. The more data available, the more robust will be the determination of features specific to the patient group. For example, a minimum 20 cells per patient may be used. For a reasonable result, 100 cells may be used for each patient group.

The discrete L-dimensional probability distribution (histogram) described in the previous paragraph can be generated for each therapy outcome that might be of interest (an example set of outcomes will be given hereinafter). The same process can be repeated for all therapies of interest. To use OPRA on new patients outside of the study pool, a new L-dimensional normalized histogram, the “fingerprint vector” can be generated for the patient.

The final complete feature vector contains all the features extracted from the analysis of protein distribution on patient cells and the spatial distribution of cells in tissues (across multiple patients from a particular outcome group). The feature vector forms the basis for the generation of the fingerprint vector which contains the features unique to the patient group.

The data vector is the M-dimensional vector based on which we get the L principal components (dimensions) for the L dimensional feature vector. L-dimensional normalized histogram is generated based on multiple dimensional feature vectors from different patients from the same disease outcome forming the fingerprint vector. A fingerprint vector is generated independently of the downstream k-means analysis. The fingerprint vector is a series of principal components of the analysed features of individual cells from e.g. a patient with a known or unknown outcome.

An L-dimensional normalized histogram, a fingerprint vector, can be generated for each patient within the study pool by normalizing the vectors of multiple patient vectors. In other words, the input is a vector from each patient in the study pool, which are normalized, and the output is a normalized fingerprint vector for each patient. In this way, a new patient vector outside of the study pool can be normalized based on the existing normalized vectors thus making it comparable.

As discussed in step 260, a further clustering algorithm or a partitioning analysis may be performed on the feature vector. In this case, a K-dimensional reference probability distribution can be built in the same way described above using the count of cells in each of the K regions in the L-dimensional PCA space as the feature vector to construct the reference histogram.

In some implementations, when the locations of the K′ clustering regions will be different for each patient, a least squares Euclidean distance minimization can be performed between a set of reference cluster centers and, for instance, affine transformations of the cluster centers from patients in the study for one specific therapy. Once the global cluster centers are known, they can be enumerated. A K′-dimensional feature vector can be calculated for a new patient by performing least squares minimization of the distance between the reference clustering centers and the clustering centers of this particular patient under affine transformations. The identity of an unknown clustering center from the new patient can be found by applying the affine transformation and selecting the closest reference cluster center.

At step 320, the fingerprint vector is classified into one of the types of the reference cell populations using an outcome prediction algorithm (OPRA).

The fingerprint vector can be compared using probability distance metrics, e.g. the Wasserstein metric, and an “outcome probability vector” can be calculated that calculates the probability of the fingerprint vector from the patient matching any of the reference probability distributions for each outcome and each treatment.

A method for obtaining the outcome probability vector based on the comparison of the reference population and the patient fingerprint vector is achievable using a statistical model called logistic regression. The model is applied sequentially (or in parallel) or in a pairwise manner to the patient fingerprint vector and the fingerprint vectors of the reference populations of the possible outcomes, which yields the probability of the respective outcome.

Classification of the cells may be carried out using a machine learning classification algorithm, such as logistic regression or a convolutional neural network (CNN). The classification algorithm can be created by fitting a training dataset using machine learning analysis, to link spatial distribution characteristics to defined classification types. A supervised learning algorithm may be used to fit the training dataset, e.g. a logistic regression algorithm. Details of such approaches are described, for example, in Deep Learning by Ian Goodfellow, Yoshua Bengio, and Aaron Courville (MIT Press, 2016), which is incorporated herein by reference, in particular section 5.7. The logistic regression algorithm may be implemented using the MatLab software package, for example by using the Machine Learning and Deep Learning application package to train the system, and analysing sample data using the mnrfit function (as described, for example at https://uk.mathworks.com/help/stats/train-logistic-regression-classifiers-in-classification-learner-app.html and https://uk.mathworks.com/help/stats/mnrfit.html).

The method provided herein may be used for application such as patient stratification and quality assessment of cell therapy products, a reference library of patient data is assembled by applying the method to data obtained from the cells of the patient. This may uniquely characterise patient tumor samples, tumor neutralizing potential of native T-cell populations in the presence of drug molecules, and therapeutic immune cells. To this end, the disclosed method may be used to analyse and categorise the expression of any cell surface marker on any cell type.

In particular implementations, the disclosed method may be used to analyse and categorise the expression of one or more surface markers on a diseased cell from a subject, for example for comparison of the diseased cell to similarly diseased cells from patients with a known therapeutic outcome, to thus provide an indication of the suitability of a particular therapeutic approach for the treatment of the disease in the subject patient.

The target diseased cell may be a cancerous cell, which may be a cell from any type of cancer, including, for example, Acute Lymphoblastic Leukemia (ALL), Acute Myeloid Leukemia (AML), Adrenocortical Carcinoma, Kaposi Sarcoma, Lymphoma, Anal Cancer, Appendix Cancer, B Cell Lymphoma, Basal Cell Carcinoma of the Skin, Bile Duct Cancer, Bladder Cancer, Bone Cancer, Brain Cancer, Breast Cancer, Bronchial Cancer, Burkitt Lymphoma, Carcinoid Cancer, Atypical Teratoid/Rhabdoid Tumor, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer (Intraocular Melanoma, Retinoblastoma), Fallopian Tube Cancer, Fibrous Histiocytoma, Gallbladder Cancer, Gastric (Stomach) Cancer, Gastrointestinal Carcinoid Tumor, Gastrointestinal Stromal Tumors (GIST), Testicular Cancer, Gestational Trophoblastic Disease, Glioma, Hairy Cell Leukemia, Head and Neck Cancer, Heart Tumors, Hepatocellular (Liver) Cancer, Histiocytosis, Hodgkin Lymphoma, Hypopharyngeal Cancer, Intraocular Melanoma, Islet Cell Tumors, Pancreatic Neuroendocrine Cancer, Kidney (Renal Cell) Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and Tracheobronchial Tumor), Lymphoma, Melanoma, Merkel Cell Carcinoma (Skin Cancer), Mesothelioma, Mouth Cancer, Multiple Myeloma/Plasma Cell Neoplasms, Mycosis Fungoides (Lymphoma), Myelodysplastic Syndromes, Myelogenous Leukemia, Myeloid Leukemia, Nasal Cavity and Paranasal Sinus Cancer, Nasopharyngeal Cancer, Neuroblastoma, Non-Hodgkin Lymphoma, Non-Small Cell Lung Cancer, Oral Cancer, Osteosarcoma and Malignant Fibrous Histiocytoma of Bone, Ovarian Cancer, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Peritoneal Cancer, Prostate Cancer, Rectal Cancer, Renal Cell (Kidney) Cancer, Retinoblastoma, Rhabdomyosarcoma, Salivary Gland Cancer, Ewing Sarcoma, Osteosarcoma, Soft Tissue Sarcoma, Uterine Sarcoma, Sézary Syndrome, Skin Cancer, Small Cell Lung Cancer, Small Intestine Cancer, Squamous Cell Carcinoma of the Skin, Squamous Neck Cancer, Stomach (Gastric) Cancer, T-Cell Lymphoma, Testicular Cancer, Throat Cancer, Nasopharyngeal Cancer, Oropharyngeal Cancer, Hypopharyngeal Cancer, Thymoma and Thymic Carcinoma, Thyroid Cancer, Urethral Cancer, Uterine Cancer, Uterine Sarcoma, Vaginal Cancer, Vulvar Cancer, or Wilms Tumor.

The surface marker may be any cell surface marker or biomarker, which may be a cell surface protein. The surface marker may be a marker that is suitable for use as a phenotypic marker to identify a particular cell type, or a particular maturation or activation state of a particular cell type. In certain cases the distribution of cytoplasmic, non-plasma-membrane bound and/or proteins confined to trafficking compartments can be considered as biomarkers.

In many cell therapies such as Chimeric Antigen Receptor (CAR) T cell therapy, biomarkers, for example on the surface of malignant cells, serve as targets for directing cytotoxic T cells. Such biomarkers may be used as target surface markers in the disclosed method.

T cells are a critical component of the adaptive immune system as they not only orchestrate cytotoxic effects, but also provide long term cellular ‘memory’ of specific antigens. A patient may have tumor-infiltrating lymphocytes specific for their tumor but these cells are often retrained within the tumor microenvironment and become anergic and non-functional. T cells endogenously require the interaction between their T cell receptor and MHC molecules in order to become activated, but CAR-T cells have been engineered to activate via a tumor-associated or tumor-specific antigen (TAA and TSA, respectively) expressed on the target cell. CAR-T cells are a “living drug” comprising a chimeric antigen receptor (CAR) which includes a targeting domain (such as a ligand or antibody fragment which binds to the TAA or TSA) fused to the signalling domain of a T cell receptor. Upon recognition and binding of the CAR to the appropriate surface marker TAA or TSA, the T cell activates and initiates cytotoxic killing of the target cell. The difficulties in designing optimal CAR-T cell therapy include on-target off-tumor cytotoxicity, persistence in vivo, immunosuppressive tumor microenvironment, and cytokine release syndrome. The disclosed method may be used to analyse and categorise both CAR-T and target cells based on surface marker expression to improve CAR-T cell development and to identify the most appropriate cells or cell therapy for administration to specific patients.

Thus, in some implementations, the disclosed method may be used to analyse and categorise potentially therapeutic cells that may be used for CAR-T cell therapy. For example, the surface marker may be a marker present on the surface of CAR-T cells, for example, that may be used to identify “naive”, “memory”, “effector” and/or “exhausted” CAR-T cells.

In particular implementations, the disclosed method may be used to analyse and categorise the expression of one or more surface markers on CAR-T cells for potential therapeutic use. For example, the disclosed method may be used to provide a comparison of the CAR-T cell to similar CAR-T cells from patients with a known therapeutic outcome, to thus provide an indication of the suitability of a particular CAR-T cell for therapeutic use in the subject patient.

The disclosed method may also be used to characterise and categorise target cells based on a particular surface (and/or intracellular) marker or markers, and may thus be used to identify patients that are likely to benefit from a particular cell therapy. For example the disclosed method may be based on the expression of CD19, a B cell marker expressed highly on malignant B cells. The method may in addition, or alternatively, be used to categorise cells on the basis of other targetable biomarkers, which may be expressed on any of a range of cancerous target cells, such as any of those listed above. Thus, in some implementations, the disclosed method may be used to categorise CAR-T cells which target one or more surface markers selected from CD19, CD20, Mesothelin, Her2, PSCA, CEA, CD33, GAP, GD2, CD5, PSMA, ROR1, CD123, CD70, CD38, BCMA, Muc1, EphA2, EGFRVIII, IL13Ra2, CD133, GPC3, EpCam, FAP, VEGFR2, CT antigens, GUCY2C, TAG-72, and HPRT1/TK1. In these particular implementations, the disease targeted by the CAR-T cell therapy may be selected from ALL, B cell lymphoma, leukemia, Non-Hodgkin lymphoma, Pancreatic cancer, Cervical Cancer, Ovarian Cancer, Lung Cancer, Peritoneal carcinoma, Fallopian tube cancer, Colorectal Cancer, Breast Cancer, CNS tumor, Gastric Cancer, Glioma, Glioblastoma, Liver metastases, Myeloid leukemia, solid tumors, sarcoma, neuroblastoma, T cell acute lymphoblastic lymphoma, T-non-Hodgkin lymphoma, Prostate cancer, Bladder cancer, AML, B cell malignancies, renal cell cancer, melanoma, myeloma, Sarcoma, hepatocellular carcinoma, AML, Liver Cancer, Heptocellular carcinoma, Lymphoma, Leukemia, Colon Cancer, Esophageal Carcinoma, Hepatic Carcinoma, and Pleural Mesothelioma.

In particular implementations, biomarkers that may be used in the disclosed method include liquid tumor markers, such as: CD5, which may be used as a CAR target to treat T cell malignancies such as T-ALL, and also B cell lymphomas; IL3Ra or CD123, which may be used as a CAR target to treat hematological malignancies including blastic plasmacytoid dendritic cell neoplasm (BPDCN), hairy cell leukemia, B-cell acute lymphocytic leukemia (B-ALL), and Acute myeloblastic leukemia (AML); CD33, which may be used as a CAR target to treat AML; CD70, which may be used as a CAR target to treat large B-cell and follicular lymphomas, Hodgkin's lymphoma, multiple myeloma, EBV-associated malignancies, glioma, breast cancer, renal cell carcinoma, ovarian cancer, and pancreatic cancer; and CD38, which may be used as a CAR target to treat myeloma; and BCMA, which may be used as a CAR target to treat myeloma.

In other implementations, biomarkers that may be used in the disclosed method include solid tumor markers, such as: Mesothelin (MSLN), which may be used as a CAR target to treat ovarian cancers, non-small-cell lung cancers, breast cancers, esophageal cancers, colon and gastric cancers, pancreatic cancers, thyroid cancer, renal cancer, and synovial sarcoma; Her2, which may be used as a CAR target to treat breast cancer, and head and neck squamous cancer; GD2, which may be used as a CAR target to treat neuroblastoma; MUC1, which may be used as a CAR target to treat breast and ovarian cancers; GPC3, which may be used as a CAR target to treat hepatocellular carcinoma, breast cancer, melanoma, pancreatic cancer, lung cancer, and colorectal cancer; IL13ra2, which may be used as a CAR target to treat glioma; PSCA, which may be used as a CAR target to treat prostate cancer, gastric cancer, gallbladder adenocarcinoma, non-small-cell lung cancer, and pancreatic cancer; VEGFR2, which may be used as a CAR target to treat squamous cell carcinomas of the head and neck, colorectal cancer, breast cancer, and NSCLC; CEA, which may be used as a CAR target to treat colorectal cancer, gastric cancer, pancreatic cancer, ovarian cancer, lung cancer, skin cancer, and NSCLC; PSMA, which may be used as a CAR target to treat prostate cancer; ROR1, which may be used as a CAR target to treat pancreatic cancer, ovarian cancer, breast cancer, lung cancer, colorectal cancer, and gastric cancer; FAP, which may be used as a CAR target to treat pleural mesothelioma; EpCAM, which may be used as a CAR target to treat bladder cancer, head and neck cancer, ovarian cancer, prostate cancer, breast cancer, and peritoneal cancer; EGFRvIll, which may be used as a CAR target to treat glioblastoma; and EphA2, which may be used as a CAR target to treat lung cancer, glioma, and glioblastoma.

In some implementations the disclosed methods may be used in relation to immune checkpoint receptors, for example, to define cellular outcome. Numerous inhibitory checkpoints to activation exist across a range of lymphocytes and myeloid cells, predominantly to regulate against autoimmunity but also to ensure appropriate cell-cell interactions. Such immune checkpoints are typically mediated by receptor-ligand associations between transmembrane proteins on the opposing surfaces of interacting cells. The presence or absence of cognate ligand on one cell therefore determines the activity of the corresponding receptors on the other, thus allowing cell-to-cell communication of immune status. Given its inhibitory nature, there is strong selective pressure amongst cancerous and precancerous cells to increase immune checkpoint activity, thereby inhibiting local immune responses and protecting against attack by tumor antigen-specific lymphocytes. Increased expression of immune checkpoint regulators is a common feature of many solid tumors, including melanoma, lung cancer, kidney cancer, and certain lymphomas. Consequently, blockade of immune checkpoints using monoclonal antibodies that interfere with checkpoint receptor-ligand interactions is a rapidly growing area of immunotherapy for a range of cancers.

The extent of inhibition emerging from checkpoint receptors is substantially affected by both their, and their ligands', nanoscale organisation. Typically, such receptors convey inhibitive effects through the recruitment of tyrosine phosphatases that are capable of dephosphorylating activatory receptors, thereby terminating their signalling. The range of such effects is inherently limited by the length of the inhibitory receptor's cytoplasmic domains, and so only immediately proximal target receptors are accessible for inhibition. Consequently, receptor clusters of different morphologies and densities will have accordingly different accessibility to target proteins. Similarly, the nature of clustering also influences the potency of each individual inhibitory receptor, since tightly clustered ligands induce more robust signalling in their cognate receptors. This is due to the increased local concentration of kinases and other interaction partners in dense clusters, which amplifies the baseline activation experienced by a lone receptor.

Thus, in particular implementations, the disclosed method may be used in connection with immune checkpoint receptor-ligand pairs. Indeed, the disclosed method may be used in connection with most, if not all, immune checkpoint receptor-ligand pairs. Examples are given below:

1. Programmed-death 1 (PD1) & PD1 ligand (PDL1). T cell-expressed PD1 and its antigen-presenting cell (APC)-expressed ligand PDL1 represent the most notable checkpoint pair that can be examined using the method described herein, OPRA (Outcome Prediction Algorithm). Engagement of PD1 by PDL1 leads to potent inhibition of T cell responses, and PD1 or PDL1 are targeted in six of the seven currently FDA-approved checkpoint blockade cancer immunotherapies. The activated behaviour of PD1 is well understood, as are its effects on signalling from activatory receptors in T cells, particularly its primary target CD28. Much of the research describing the dependence of inhibitory effects on molecular reach was performed on PD1, and the formation of activation-dependent PD1 clusters is well established. Thus, PD1 and PDL1 may be used as target surface markers in the disclosed method.

2. Cytotoxic T lymphocyte-associated protein 4 (CTLA4). CTLA4 on T cells engages B7 proteins CD80 and CD86 on APCs and promotes termination of T cell activation in response to antigen. This is mediated in part due to competition with CD28 for B7 engagement, and the close proximity of CTLA4-recruited tyrosine phosphatases to CD28, while the clustering behaviour of CTLA4 is also known to be strongly affected by the extent of activation. CTLA is the target of the FDA-approved checkpoint inhibitor Ipilimumab. Thus, combinations of CTLA4 with CD80, and/or CD86 may be used as target surface markers in the disclosed method.

3. T cell-immunoglobulin and mucin-domain containing 3 (Tim3). Tim3 is an inhibitory receptor highly expressed on tumor-infiltrating lymphocytes that is activated in response to binding its receptor galectin-9 on APCs. It is particularly prominent in the exhaustion of cytotoxic T cells, and hence significant in regulating anti-tumor responses. Inhibitory signalling from Tim3 interacts with that from PD1, and hence a number of Tim3-blocking monoclonal antibody therapies are currently in clinical trials in combination with anti-PD1/PDL1 treatment (e.g. MBG453, TSR-022). Thus, Tim3 and galectin-9 may be used as target surface markers in the disclosed method.

4. B- and T-lymphocyte-attenuator (BTLA). BTLA is activated by its ligand HVEM (Herpesvirus entry mediator), whereupon it preferentially inhibits signalling through the TCR. Such inhibition is strongly dependent on the close association of BTLA- and TCR-containing protein clusters, and the nature of BTLA clustering is strongly associated with the extent of inhibition. BTLA is also able to bind in cis to T cell-expressed HVEM, the extent of which will alter its availability to APC-presented HVEM and so influence clustering. Several BTLA-blocking monoclonal antibody therapies are currently in development. Thus, BTLA and HVEM may be used as target surface markers in the disclosed method.

In some implementations, one or a combination of immune checkpoint regulators on the surface of a single cell type may be investigated using the disclosed method. For example, one or a combination of immune checkpoint regulators (such as immune checkpoint receptor ligands) may be used as target surface markers on target cells, such as cancerous or suspected cancerous cells, in the disclosed methods, to determine how best to target the cells with an immune checkpoint receptor therapy.

In another example, one or a combination of immune checkpoint regulators (such as immune checkpoint receptors) may be used as target surface markers on the surface of one or more candidate effector cell types, such as different T cells, in the disclosed methods, to analyse and categorise potentially therapeutic cells that may be used in a specific immune checkpoint receptor therapeutic treatment.

Although best-described in the context of immune checkpoint inhibition, these receptors are also all of clinical relevance in the field of chimeric antigen receptor (CAR)-T cell therapy since their clustering behaviour in vitro provides predictions for their in vivo activity. Determination of clustering properties is also highly relevant for CARs themselves, particularly several of the most recently generated versions that combine complex regulatory strategies with antigen-specificity. The activity of avidity-controlled CARs, for example, is inherently determined by their nanoscale organisation, which can be influenced both by ligand-clustering and small-molecule intervention. There are also a wide range of bi-specific CAR-T therapies in development, for which the relative nanoscale organisation of the different CARs and/or different CAR ligands will heavily impact the degree of activation. The expansion of this concept into logic-gating CARs further increases the potential importance of information, as provided by the disclosed method, regarding CAR nanoscale clustering in the prediction of clinical outcomes.

### Example 1

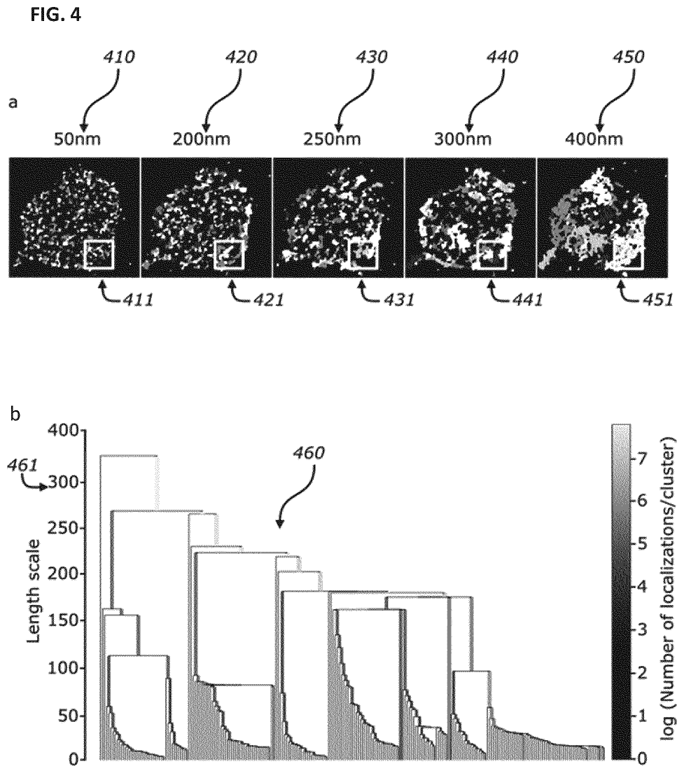

FIG. 4a shows an image that illustrates the clusters defined on a cell defined at various length scales.

Direct Stochastic Optical Reconstruction Microscopy (dSTORM) was used to detect protein on a cell.

The proteins were stained using directly conjugated (Alexa Fluor 647 or Alexa Fluor 555) or non-conjugated primary antibodies. For the latter, fluorescently labelled secondary antibodies were used. In order to achieve photoblinking a thiol based reducing buffer with an oxygen scavenger was used. A minimum of 10000 frames were acquired using the Nanoimager S (ONI, Oxford Nanoimaging) with the following specifications: lasers 405 nm (150 mW), 473 nm (1 W), 561 nm (1 W), 640 nm (1 W), dual emission channels split at 640 nm. The super-resolved images were reconstructed in NimOS (ONI). The dSTORM data, namely the set of coordinates of the fluorescently labelled molecules in the sample, was filtered based on number of photons (set to a minimum of 500), localization precision (15 nm x/y) and sigma value (200 nm x/y).

For example, after the molecules of interest are detected and localised to yield molecular coordinates using a suitable detection technique such as dSTORM (steps 110 and 120), a spatial distribution analysis algorithm, such as HDBSCAN analysis, is applied to the molecular coordinates to identify clustering of the spatial coordinates (step 210). For example, the evaluation of the protein clustering can be performed using HDBSCAN algorithm in a Python environment where the minimum number of points per cluster was set to 5. The input of this algorithm is a list of spatial 2D or 3D coordinates with metadata for each point, and the output is a hierarchical data structure that describes for each localization a series of N cluster names which the point belongs to at O different spatial scales, where O can differ between localizations, where N and O are positive integers. In other words, localizations belong to different clusters at different length scales. Groups of localizations can belong to different number of clusters based on their spatial distribution on the cell surface.

Clusters at different length scales contain varying amounts of localizations that has an effect on the amount of localizations and clusters which are considered noise. Therefore, noise can also be considered for extracting relevant information. The data used to generate FIG. 4a contains a minor amount of noise due to pre-filtering of localizations prior to HDBSCAN. However, the definition of noise (localizations in vs. not in a particular cluster) may change with the length scale.

The data vector had 50 dimensions and contained the following properties: 1. number of clusters, 2. mean, median, SD of area of clusters, 3. mean, median, SD of distance between clusters, 4. mean, median, SD of number of localizations per cluster, at spatial scales 10 nm, 50 nm, 100 nm, 500 nm, 1000 nm.

Panels, 410, 420, 430, 440, 450, labelled as “50 nm,” “200 nm,” “250 nm,” “300 nm,” “400 nm,” shows the clusters of the spatial coordinates of the protein of interest at respective length scales (FIG. 4a). The default/standard HDBSCAN is used to detect clusters. The only input parameter which is needed for running HDBSCAN is the minimum number of localizations per cluster. This was set to minimum of 5 which refers to the minimum number of localizations that is needed for a cluster to be considered a cluster. Additional settings are the selected length scales at which the hierarchical cluster data exemplified in FIG. 4 is sampled.

FIG. 4b shows a graph that illustrates a HDBSCAN cluster tree.

A graph 460 shows a HDBSCAN cluster tree generated based on a representative region of interest 411, 421, 431, 441, 451, delineated as a square within each panel 410, 420, 430, 440, 450. This provides an alternative visualization of cluster distribution, number and localization number per cluster at specified length scales.

A vertical axis 461 of the graph 460 represents the length scale.

Localizations may belong to different clusters at different length scales. Groups of localizations can belong to a different number of clusters based on their spatial distribution on the cell surface. This is shown in the graph 460: a major split is visible at the highest spatial scale. This divides the localizations into two clusters initially. The localizations belonging to the branch on the left show different clustering (branching points) at various length scales compared to the rest of the localizations (belonging to the branch on the right).

In Examples 2 and 3, a data vector was constructed based on the data obtained from the spatial distribution analysis (step 220). The data vector had 50 dimensions and contained the following properties:
1. number of clusters;
2. mean, median, variance of area of clusters;
3. mean, median, variance of distance between clusters;
4. mean, median, variance of number of localizations per cluster, at spatial scales 10 nm, 50 nm, 100 nm, 500 nm, 1000 nm.

### Example 2

This example demonstrates the use of the disclosed method to determine the most appropriate therapy for an individual cancer patient. For example, the most appropriate therapy may be therapy with the greatest likelihood of achieving remission for the patient with the fewest side effects.

FIG. 5a is a table which illustrates an example of classification of a test patient's tumor sample based on data obtained from reference patient samples according to the outcomes from multiple therapeutic strategies data (referred to as “therapies”).

The table 500 shows an example of a predicted level of tumor responsiveness of a test patient to three different oncological therapies:
1. a checkpoint therapy 540;
2. a CAR-T therapy 550; and
3. a chemotherapy 560.

The test patient sample is a tumor sample taken from the test patient and the reference patient data is produced from samples of the same type of tumor obtained from each patient in a reference patient population, wherein each patient in the reference patient population has undergone one of the different therapies, and the clinical outcome of that therapy has been determined.

From the samples obtained from the reference patients, multiple distinct populations of clinical outcomes can be identified for each therapy. These identified populations are referred to as “reference patient groups”.

The different reference patient groups, 18 in total, are shown in FIG. 5a. The clinical outcome for each therapy 540, 550, 560 is divided into two groups, namely a first group 510 representing ‘malignant with minimal or no reduction of tumor cells’ and a second group 520 representing ‘malignant with strong reduction of tumor cells’ 520.

The second group 520 is divided into a first subgroup 521, representing ‘complete remission’ and a second subgroup 522, representing ‘temporary remission.’

The first group 510, the first subgroup 521, and the second subgroup 522, are each respectively further divided into two alternatives representing ‘strong side effects’ and ‘minimal or no side effects.’

Therefore, for each therapy, 540, 550, 560, the reference patients are divided into 6 clinical outcomes, i.e. 6 reference patient groups.

As discussed in step 310 and according to the methods described in FIGS. 1 and 2, a first spatial organisation is characterised from the tumor sample of the test patient, and a second spatial organisation can be characterised for each clinical outcome of a particular therapy 540, 550, 560, based on the data vectors of the reference patient groups. The second spatial organisation may be in the form of probability distribution or a histogram in the reduced dimensional space, given by, for example, the Principal Component Analysis.

Results of the first spatial organisation for 3 different test patients (i.e. Patients 1-3) are shown in FIG. 5b.

FIG. 5b shows a first graph 570, a second graph 580 and a third graph 590, corresponding to the exemplary results of the method 200 performed on the data vectors of the three different patients.

As discussed above, the data from each cell is constructed into a 50-dimensional data vector. The 50-dimensional data vector for each cell is reduced to a 2-dimensional vector via dimension reduction analysis, in this case, the Principal Component Analysis.

The result of the dimension reduction analysis on the data vector, a 2-dimensional data vector, corresponds to a coordinate in a 2-dimensional plane spanned by the principal components. This is referred to as a “reduced data vector” for convenience. The axes of the graphs 570, 580, 590 are labelled as ‘PC1’ and ‘PC2’, representing a first principal component and a second principal component.

Each dot in the graphs 570, 580, 590 represents the reduced data vector from a single patient tumor cell.

A further partitioning analysis is applied to the collection of the reduced data vectors. In the example of FIG. 5b, k-means clustering is performed with K=5 such that the reduced data vectors are grouped into 5 subgroups.

The different data labels indicate the detected cell clusters (1-5).

As discussed in steps 320 and 330 of FIG. 3, the first spatial organisation for a test patient (for example, patient 1, 570) and the second spatial organisation are compared, namely by evaluating the probability distance between the first spatial organisation and the second spatial organisation. Based on the evaluated probability distance, which ranges from 0 to 1, the likelihood of the test patient being classified into one of the 18 reference patient groups is determined.

In the example of FIG. 5a, in relation to the checkpoint therapy 540, a first outcome 541 is predicted to be the most likely outcome, with probability distance 0.52. This outcome corresponds to the second group 520 and second subgroup 522, i.e. an outcome of malignant with strong reduction of tumor cells, temporary remission, with minimal or no side effects.

In relation to the CAR-T therapy 550, a second outcome 551 is predicted to be the most likely outcome, with probability distance 0.59. This outcome corresponds to the second group 520 and first subgroup 521, i.e. an outcome of malignant with strong reduction of tumor cells, complete remission, with minimal or no side effects.

In relation to the chemotherapy 560, a third outcome 561, is predicted to be the most likely outcome, with probability distance 0.6. This outcome corresponds to the first group 510 and first subgroup 521, i.e. an outcome of malignant with minimal to no reduction of tumor cells, with strong side effects.

Thus, in this example, the most appropriate therapy for the test patient would appear to be CAR-T therapy, because based on a comparison with the reference patient data the most likely outcome of CAR-T therapy for test patient is complete remission with minimal or no side effects.

This example shows that based on a comparison with data from reference patients having similar tumors and known therapeutic outcomes, the disclosed method can be used to determine the most appropriate therapy for the test patient, for example, the therapy with the greatest likelihood of achieving remission for the test patient with the fewest side effects.

### Example 3

This example demonstrates a method for the identification of subpopulations of engineered immune cells for the purpose of refining their production and improving their efficacy. This process is currently monitored according to the number of cells expressing markers for “naive”, “memory”, “effector” and “exhausted” CAR-T cells.

The expression and spatial distribution patterns of the CAR on established “naive”, “memory”, “effector”, “exhausted” marker expressing cells refines the understanding of what can be considered the ideal population of CAR T-cells in terms of efficacy. The workflow for identifying these populations is similar to that described above in Example 2, in relation to the patient tumor samples.

Once sufficient reference sample populations are analysed, the robustness of CAR-T state (efficacy) determination based on the CAR expression can be increased. By applying the method described for post-transformation T-cells (CAR-Ts), a pre-transformation analysis of patient T-cells can also be achieved.

By looking at the distribution of native T-cell receptors in populations expressing the mentioned markers for “naive”, “memory”, “effector” and “exhausted” T cells, a prediction can be made on post transformation efficacy, making this a crucial step in the decision whether the patient is eligible for autologous CAR-T therapy.

FIG. 6a is a table which illustrates an example of the classification of transformed T cells into subpopulations, based on data obtained from reference populations of T cells that have undergone a transduction procedure aimed at inducing CAR expression.

The table 600 illustrates an example of predicted outcome of three different populations of T cells that have been obtained from the same patient and transduced to express CAR. The three different populations of T cells are referenced as CAR-T1 640, CAR-T2 650, and CAR-T3 660.

The three populations of T cells are obtained from the same patient and transduced independently with CAR. The reference T cell population data is produced from samples of T cells similar to the test cells that have been transduced with CAR in an identical procedure, and wherein the outcome of the expression of CAR and the properties of the transduced cells have previously been determined.

Multiple distinct outcomes can be identified in the reference T cell population data, for example, according to the expressions of the CAR, and/or other mentioned surface markers such as phenotypic markers for “naive”, “memory”, “effector” and/or “exhausted” T-cells. These identified populations, are referred to here as “reference T cell groups”

Different reference T cell groups, 5 in total, are shown in FIG. 6a. The reference T cells groups are divided into two groups primary categories on the basis of CAR expression, namely a first group 610 where CAR is not expressed by the transduced T cells, labelled as ‘transduced patient T cells do not express the CAR’ and a second group 620 where CAR expression on the T cells is observed, labelled as ‘transduced T cells express the CAR’.

The first group 610 is not further subdivided.

The second group 620 is divided according to whether the CAR-Ts can be expanded or not, namely into a first subgroup 621, in which the CAR-Ts cells are capable of expansion, labelled as ‘CAR-Ts can be expanded’ and a second subgroup 622, in which the CAR-T cells are incapable of expansion, labelled as ‘CAR-Ts cannot be expanded.’

The second subgroup 622 is not further subdivided.

The first subgroup 621 is further divided into two groups based on whether or not the CAR T cells will become exhausted. T-cell exhaustion refers to a state of cellular dysfunction characterised, for example, by a reduction in the release of effector molecules and/or an increase in the expression of inhibitory receptors. These groups are labelled as ‘majority of CAR-Ts will become exhausted’ and ‘majority of CAR-Ts will not become exhausted.’

The latter group, in which T-cell exhaustion is not observed in the majority of cells CAR-Ts are not exhausted, is further divided into two groups according to whether or not Cytokine Release Syndrome (CRS) may be observed in the recipient following the administration of the CAR T-cells. CRS is a potentially life-threatening, systemic inflammatory response. These further groups are labelled as ‘CAR-Ts cause CRS’ and ‘CAR-Ts do not cause CRS’, respectively.

In total, therefore, there are 5 reference T cell groups.

For both reference cells and each the three batches of the test patient cells CAR-T1, T2, and T3, following transduction of the cells, the spatial coordinates of the CAR and/or other surface markers on the surface of the T cell are obtained.

After performing the spatial distribution analysis algorithm data vectors are constructed.

A first spatial organisation is characterised from each batch of the CAR-T cells of the test patient, as discussed in step 310 and according to the methods 100, 200 described in FIGS. 1 and 2. The example results of the first spatial organisation are shown in FIG. 6b.

From the data vectors of the reference cells, a second spatial organisation can be characterised as discussed in step 310, and according to the methods described in FIGS. 1 and 2. The second spatial organisation may be in the form of probability distribution or a histogram in the reduced dimensional space, given by, for example, Principal Component Analysis.

FIG. 6b shows a first graph 670, a second graph 680 and a third graph 690, corresponding to the results of a dimension reduction analysis and a partitioning analysis on the data vectors obtained from the first batch 640, the second batch 650, the third batch 660 of the CAR-T cells of the test patient, respectively.

As discussed above, the data from each cell is constructed into a 50-dimensional data vector. The 50-dimensional data vector for each cell is reduced to a 2-dimensional vector via the dimension reduction analysis, in this case, the Principal Component Analysis.

The result of the dimension reduction analysis on the data vector, a 2-dimensional data vector, corresponds to a coordinate in a 2-dimensional plane spanned by the principal components. This is referred to as a “reduced data vector” for convenience.

The axes of the graphs 670, 680, and 690 are labelled as ‘PC1’ and ‘PC2’, representing a first principal component and a second principal component.

Each dot in the graphs 670, 680, 690 represents the reduced data vector from a single transduced T cell of the test patient.

As explained in step 240, a further partitioning analysis is applied to the collection of the reduced data vectors. In the example of FIG. 6b, k-means clustering is performed with K=5 such that the reduced data vectors are grouped into 5 subgroups.

The different data labels indicate detected cell clusters within a specific CAR-T population (1-5).

As discussed in FIG. 3, based on the evaluated probability distance, which ranges from 0 to 1, each batch of the CAR-T cells of the test patient, 640, 650, 660 is classified into one of the 5 reference T cell groups.

In the example of FIG. 6a, in relation to the first batch 640 ‘CAR-T 1’, a first outcome 641 is predicted to be the most likely outcome with probability distance 0.7. This outcome corresponds to the first group 610 where transduced T cells do not express the CAR.

In relation to the first batch 650 ‘CAR-T 2’, a second outcome 642 is predicted to be the most likely outcome with probability distance 0.7, indicating that the T cells express the CAR and can be expanded, but the majority of CAR-Ts will become exhausted.

In relation to the first batch 660 ‘CAR-T 3’, a third outcome 643 is predicted to be the most probable likely outcome with probability distance 0.65, indicating that the T cells express the CAR and can be expanded, will not become exhausted and should not cause CRS.

Ultimately the information from the three reference databases (patient tumor samples, patient T cells and CAR-Ts) can be used to predict the therapeutic outcome based on the detected populations of engineered immune cells and the detected populations of cells in a patient diagnosed with a specific case of malignancy.

The procedures described in FIGS. 5 and 6 allows the detection of multiple distinct populations of therapeutic immune cells such as CAR-Ts or clinical outcomes based on patient samples relying on the methods and parameters described in FIGS. 1 to 3. The identified populations serve as references for the evaluation, classification and quantification of patient and therapeutic cell phenotypes associated with:
1. CAR-T maturity and efficacy based on CAR expression, distribution, molecular organization and T-cell state; and
2. Tumor responsiveness to immunotherapy (monotherapy, combination therapy, engineered immune cell therapy i.e. CAR-T) according to the expression, distribution and molecular organization of tumor markers (such as CTLA-4, PD-1, PD-L1, CD19, CSF1R).

The different patient tumor cell phenotypes may be more or less susceptible to treatment by immunotherapy, hence the importance of quantitatively distinguishing these phenotypes.

FIG. 7 is a flowchart that illustrates a method of classifying a cell.

At step 710, proteins on or within the cell are detected at a single-molecule level.

At step 720, the distribution and the clusters of the detected molecules are investigated.

At step 730, the distribution of the cells and the interaction between the cells are investigated.

At step 740, a feature vector is constructed containing information at multiple spatial scales.

At step 750, a dimension reduction analysis.

At step 760, a normalized L-dimensional histogram, a fingerprint vector, is constructed based on the data of patients from within and outside a study pool.

At step 770, an outcome prediction algorithm is performed to predict the outcome.

It will be understood that the present invention has been described above by way of example only. The examples are not intended to limit the scope of the invention. Various modifications and embodiments can be made without departing from the scope and spirit of the invention, which is defined by the following claims only.

All references referred to herein are hereby incorporated by reference.

Each and every compatible combination of the embodiments described herein is explicitly disclosed herein, as if each and every combination was individually and explicitly recited. Additionally, where used herein, “and/or” is to be taken as a specific disclosure of each of the two specified features with or without the other.

Unless context dictated otherwise, the descriptions and definitions of the features set out herein are not limited to any particular aspect or embodiment and apply equally to all aspects and embodiments which are described where appropriate.

1. A method of investigating a plurality of cells, comprising:
detecting one or more species of proteins on each of the plurality of cells;
obtaining respective spatial coordinates of the detected proteins within the plurality of cells;
detecting boundaries of the plurality of cells; and
constructing a data vector based on the obtained spatial coordinates and the detected boundaries.

2. The method of claim 1,
wherein constructing the data vector comprises:
performing a spatial distribution analysis algorithm such that the obtained spatial coordinates are partitioned into one or more clusters at a predetermined number of length scales,
wherein at each length scale, each cluster comprises the spatial coordinates of the detected proteins within an area corresponding to the length scale.

3. The method of claim 1,
wherein constructing the data vector comprises:
performing a spatial distribution analysis algorithm such that the obtained spatial coordinates are partitioned into one or more clusters at a predetermined number of length scales, wherein at each length scale, each cluster comprises the spatial coordinates of the detected proteins within an area corresponding to the length scale; and
determining a set of properties for the clusters at each of the length scales;
wherein the data vector comprises the set of properties determined for the clusters at each of the length scales

4. The method of claim 1,
wherein obtaining the boundaries comprises:
obtaining an optical image of the plurality of cells;
performing a segmentation algorithm on the optical image of the plurality of cells; and
extending a border obtained by the segmentation algorithm by a predetermined distance.

5. The method of claim 4,
wherein constructing the data vector further comprises:
performing colocalization analysis on an overlapping area between any two of the plurality of cells.

6. The method of claim 1, further comprising:
constructing a feature vector by performing a dimension reduction analysis on the constructed data vector,
wherein a first dimension of the feature vector is larger than two and smaller than a second dimension of the data vector.

7. The method of claim 6,
wherein the dimension reduction analysis comprises Principal Component Analysis, PCA such that the feature vector comprises a first number of principal components obtained from the data vector, and
wherein the first dimension is the first number.

8. The method of claim 1, wherein the step of detecting the one or more species of proteins on each of the plurality of cells and obtaining respective spatial coordinates comprises carrying out single molecule localisation microscopy.

9. A method of classifying a sample of cells of a patient into one or more defined types, comprising:
investigating the sample of cells of the patient using a method according to claim 1 to obtain a sample feature vector;
providing reference data, wherein the reference data comprises one or more reference feature vectors obtained for reference cells of said one or more defined types;
carrying out data analysis, comprising comparing the sample feature vector with said reference feature vector(s), and determining, based on the comparison, whether the sample of cells is classified into one of said defined types, and if so, which of the defined types.

10. The method of claim 9, wherein the one or more reference feature vectors are obtained by investigating the reference cells using the method of claim 1.

11. The method of claim 9, wherein said investigating the sample of cells of the patient uses a method according to claim 6 or claim 7.

12. The method of claim 9, wherein the reference cells of said one or more defined types correspond to diseased cells from patients which are confirmed to be responsive to a specific medical treatment.

13. The method of claim 9, wherein the reference cells of said one or more defined types correspond to therapeutic cells confirmed to achieve a specific medical outcome.

14. The method of claim 13, wherein the therapeutic cells are CAR-T cells.

15. The method of claim 13, wherein the one or more species of proteins detected includes CAR.

16. The method of claim 13, wherein the one or more species of proteins detected correspond to one or more of (i) a surface marker for naive T cells (ii) a surface marker for memory T cells, (iii) a surface marker for effector T cells (iv) a surface marker for exhausted T-cells.

17. A method of identifying the suitability of a specific medical treatment for treating a patient suffering from a disease, wherein the method involves:
investigating a sample of cells of the patient using a method according to claim 1 to obtain a sample feature vector;
providing reference data, wherein the reference data comprises one or more reference feature vectors obtained for reference cells, the reference cells corresponding to diseased cells from patients which are confirmed to be responsive to the specific medical treatment; and
carrying out data analysis, comprising comparing the sample feature vector with said reference feature vector(s), and determining the similarity of the sample of cells to the reference cells, wherein a greater degree of similarity is indicative of a greater suitability of the specific medical treatment for treating the disease.

18. A method according to claim 17, wherein the disease is cancer.

19. A method according to claim 18, wherein the specific medical treatment is selected from chemotherapy, checkpoint therapy or CAR-T cell therapy.

20. A method according to claim 19, wherein the specific medical treatment is CAR-T cell therapy.

21. A method according to claim 18, wherein the one or more species of proteins detected in the investigation step are selected from CTLA-4, PD-1, PD-L1, CD19, and CSF1R.

22. A method according to claim 17, wherein the method involves identifying a suitable medical treatment for the patient from a range of different specific medical treatments, and wherein the reference data comprises a plurality of reference feature vectors each relating to reference cells confirmed to be responsive to one of the multiple specific medical treatments.

23. A method of identifying whether a sample of T cells from a patient is suitable for use as therapeutic cells in CAR-T cell therapy, comprising
investigating the sample of cells using a method according to claim 1 to obtain a sample feature vector;
providing reference data, wherein the reference data comprises one or more reference feature vectors obtained for reference cells, wherein the reference cells are CAR-T cells from patients with a known therapeutic outcome;
carrying out data analysis, comprising comparing the sample feature vector with said reference feature vector(s), and determining the similarity of the plurality of cells to the reference cells, wherein a greater degree of similarity is indicative of a greater suitability for use in CAR-T cell therapy.

24. The method of claim 23, wherein the one or more species of proteins detected includes CAR.

25. The method of claim 23, wherein the one or more species of proteins detected correspond to one or more of (i) a surface marker for na″fve T cells (ii) a surface marker for memory T cells, (iii) a surface marker for effector T cells (iv) a surface marker for exhausted T-cells.

26. The method of claim 9, wherein the data analysis involves
evaluating a probability distance metric between the sample feature vector and the reference feature vector; and
determining whether the patient is classified into one of the defined types.

27. The method of claim 26,
wherein the data analysis further comprises:
constructing a first probability distribution from the sample feature vector and a second probability distribution from the reference feature vector,
wherein constructing the reference probability distribution comprises:
discretising respective reference feature vector of the reference cells;
and constructing a normalised histogram.

28. The method of claim 26,
when the probability distance metric between the sample of cells of the patient and one of the reference cells, is larger than a predetermined threshold, classifying the cell into the corresponding type of the reference cells.

29. The method of claim 9,
wherein data analysis further comprises:
performing a partitioning analysis on the reference feature vector such that a PCA space defined by the principal components is partitioned into a second number of regions.

30. The method of claim 31, Wherein the partitioning analysis comprises k-means clustering.

31. A method of treating a patient suffering from cancer, comprising:
investigating a sample of cells of the patient using a method according to claim 1 to obtain a sample feature vector;
providing reference data, wherein the reference data comprises at least two reference feature vectors selected from the following categories:
(i) a reference feature vector obtained for reference cells from a patient suffering from the same cancer which are confirmed to be responsive to a chemotherapy;
(ii) a reference feature vector obtained for reference cells from a patient suffering from the same cancer which are confirmed to be responsive to CAR-T cell therapy; or
(iii) a reference feature vector obtained for reference cells from a patient suffering from the same cancer which are confirmed to be responsive to checkpoint therapy;
carrying out data analysis, comprising comparing the sample feature vector with said reference feature vectors and calculating the degree of similarity between the sample vector and each reference feature vector;
selecting a reference feature vector having a degree of similarity satisfying a predetermined criterion; and
treating the patient with the same therapy as the selected reference feature vector.
